(19) United States
(12) Patent Application Publication
(10) Pub. No.: US 2026/0033752 A1
(43) Pub. Date: Feb. 5, 2026

(54) SYSTEMS, DEVICES, AND METHODS FOR ANALYTE MONITORING

(71) Applicant: ABBOTT DIABETES CARE INC., Alameda, CA (US)

(72) Inventor: Vivek S. Rao, Alameda, CA (US)

(21) Appl. No.: 19/354,529

(22) Filed: Oct. 9, 2025

Related U.S. Application Data
(63) Continuation of application No. 18/078,218, filed on Dec. 9, 2022.
Application No. 18/078,218 is a continuation of application No. 17/865,843, filed on Jul. 15, 2022, now abandoned.
(60) Provisional application No. 63/222,851, filed on Jul. 16, 2021.

(51) Int. Cl. A61B 5/145

(52) U.S. Cl. CPC A61B 5/14532; A61B 2560/063

(57) ABSTRACT

Disclosed herein are various embodiments of sensor applicator assemblies for delivering sensor control devices, wherein the embodiments include features for improving the longevity of the sensor applicator or sensor control device, as well as reducing the likelihood of mechanical failure of certain components. Some embodiments include, for example, a pull-tab coupled with the sensor or battery, an adhesive liner for the sensor control device, one or more magnets for retaining the sensor control device in the sensor carrier, and a leaf spring retraction mechanism.

### CROSS-REFERENCE TO RELATED APPLICATIONS

This application is a continuation of U.S. application Ser. No. 18/078,218, filed Dec. 9, 2022, which is a continuation of U.S. application Ser. No. 17/865,843, filed Jul. 15, 2022, now abandoned, which claims priority to U.S. Provisional Application No. 63/222,851, filed Jul. 16, 2021, which is herein expressly incorporated by reference in its entirety for all purposes.

### FIELD

The subject matter described herein relates generally to systems, devices, and methods for in vivo analyte monitoring.

### BACKGROUND

The detection and/or monitoring of analyte levels, such as glucose, ketones, lactate, oxygen, hemoglobin AIC, or the like, can be vitally important to the health of an individual having diabetes. Patients suffering from diabetes mellitus can experience complications including loss of consciousness, cardiovascular disease, retinopathy, neuropathy, and nephropathy. Diabetics are generally required to monitor their glucose levels to ensure that they are being maintained within a clinically safe range, and may also use this information to determine if and/or when insulin is needed to reduce glucose levels in their bodies, or when additional glucose is needed to raise the level of glucose in their bodies.

Growing clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

To increase patient adherence to a plan of frequent glucose monitoring, in vivo analyte monitoring systems can be utilized, in which a sensor control device may be worn on the body of an individual who requires analyte monitoring. To increase comfort and convenience for the individual, the sensor control device may have a small form-factor, and can be assembled and applied by the individual with a sensor applicator. The application process includes inserting a sensor using an applicator or insertion mechanism, such that the sensor comes into contact with a bodily fluid. The sensor control device may also be configured to transmit analyte data to another device, from which the individual or her health care provider (“HCP”) can review the data and make therapy decisions.

While current sensors can be convenient for users, they are also susceptible to malfunctions and/or mechanical failures due to improper handling and/or storage of the sensor and/or applicator, user error, lack of proper training, poor user coordination, overly complicated procedures, and other issues. This can be particularly true for analyte monitoring systems having in vivo analyte sensors used to measure an analyte level in an interstitial fluid (“ISF”), and which are inserted using sharps (also known as “introducers” or “needles”). Some prior art systems, for example, may utilize certain mechanisms and features that are susceptible to failure or reduced efficacy due to adverse conditions. These challenges and others described herein can lead to improperly inserted or damaged sensors, and consequently, a failure to properly monitor the patient's analyte level.

Thus, needs exist for more reliable sensor insertion devices, as well as systems and methods relating thereto, that are easy to use by the patient, less prone to error, and less susceptible to malfunctions or mechanical failures.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter is directed to an applicator for delivering a sensor control device. The applicator can include a housing configured to move between a first position and a second position; a sheath slidably coupled with the housing; a sharp carrier coupled with a sharp, a sensor control device comprising an analyte sensor coupled with sensor electronics, and a sensor carrier.

In some embodiments, the applicator can include an applicator cap threadably coupled with the housing, wherein the applicator cap and housing define an interior space, and wherein the interior space comprises a lightly pressurized inert gas.

According to some embodiments, the sharp carrier of the applicator can comprise one or more magnets, wherein the sensor control device further comprises one or more ferromagnetic components, and wherein the one or more magnets are configured to exert a magnetic force upon the ferromagnetic components in a proximate direction such that the sensor control device is retained in the sensor carrier when the housing is in the first position.

In some embodiments, the sensor control device can include a connector assembly comprising a pull-tab, wherein the pull-tab is comprised of an electrically insulative material, and wherein the pull-tab is removably engaged with a plurality of sensor contacts of the analyte sensor. In other embodiments, the pull-tab can be coupled with a power supply in the sensor control device. According to some embodiments, the sensor control device can further comprise an adhesive liner coupled with a bottom surface of an adhesive patch on the bottom surface of the sensor control device.

In some embodiments, the applicator can further comprise a leaf spring coupled with a sharp, wherein the sharp is configured to position at least a portion of the analyte sensor under a skin surface when the housing is moved to the second position, and wherein the leaf spring is configured to retract the sharp into the applicator after the housing is moved to the second position.

Other systems, devices, methods, features and advantages of the subject matter described herein will be or will become apparent to one with skill in the art upon examination of the following figures and detailed description. It is intended that all such additional systems, devices, methods, features, and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of a sensor applicator, reader device, monitoring system, network, and remote system.

FIG. 2A is a block diagram depicting an example embodiment of a reader device.

FIGS. 2B and 2C are block diagrams depicting example embodiments of sensor control devices.

FIGS. 3A-3G are progressive views of an example embodiment of the assembly and application of an in vivo analyte monitoring system incorporating a two-piece architecture.

FIG. 4A is a side view depicting an example embodiment of an applicator device coupled with a cap.

FIG. 4B is a side perspective view depicting an example embodiment of an applicator device and cap decoupled.

FIG. 4C is a perspective view depicting an example embodiment of a distal end of an applicator device and electronics housing.

FIG. 4D is a top perspective view of an exemplary applicator device in accordance with the disclosed subject matter.

FIG. 4E is a bottom perspective view of the applicator device of FIG. 4D.

FIG. 4F is an exploded view of the applicator device of FIG. 4D.

FIG. 4G is a side cutaway view of the applicator device of FIG. 4D.

FIG. 5A is a proximal perspective view depicting an example embodiment of a sensor carrier.

FIG. 5B is a distal perspective view depicting an example embodiment of a sensor carrier.

FIG. 5C is a top perspective view of a sensor carrier in accordance with the disclosed subject matter.

FIG. 5D is a bottom view of the sensor carrier of FIG. 5C.

FIG. 6A is a perspective view of a sharp carrier in accordance with the disclosed subject matter.

FIG. 6B is a side cutaway view of the sharp carrier of FIG. 6A.

FIG. 6C is a perspective view of a sharp carrier in accordance with the disclosed subject matter.

FIG. 6D is a side cutaway view of the sharp carrier of FIG. 6C.

FIG. 7 is a side view of an exemplary sensor according to one or more embodiments of the disclosure.

FIGS. 8A and 8B are isometric and partially exploded isometric views of an example connector assembly, according to one or more embodiments.

FIG. 8C is an isometric bottom view of the connector of FIGS. 8A-8B.

FIG. 8D and 8E are isometric and partially exploded isometric views of another example connector assembly, according to one or more embodiments.

FIG. 8F is an isometric bottom view of the connector of FIGS. 8D-8E.

FIG. 9A and 9B are side and isometric views, respectively, of an example sensor control device, according to one or more embodiments of the present disclosure.

FIGS. 10A-10E illustrate cross-sectional views depicting an example embodiment of an applicator during various stages of deployment.

FIGS. 11A-11C are side views of a leaf spring and sharp assembly according to one or more embodiments of the present disclosure.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Generally, embodiments of the present disclosure include systems, devices, and methods for the use of analyte sensor insertion applicators for use with in vivo analyte monitoring systems. An applicator can be provided to the user in a sterile package with an electronics housing of the sensor control device contained therein. According to some embodiments, a structure separate from the applicator, such as a container, can also be provided to the user as a sterile package with a sensor module and a sharp module contained therein. The user can couple the sensor module to the electronics housing, and can couple the sharp to the applicator with an assembly process that involves the insertion of the applicator into the container in a specified manner. In other embodiments, the applicator, sensor control device, sensor module, and sharp module can be provided in a single package. The applicator can be used to position the sensor control device on a human body with a sensor in contact with the wearer's bodily fluid. Some embodiments provided herein are improvements to maintain the sterility of the applicator, sensor control device, and/or analyte sensor during storage. Some embodiments provided herein reduce the susceptibility of the applicator, sensor control device, and/or analyte sensor to malfunction and mechanical failure. Other improvements and advantages are provided as well. The various configurations of these devices are described in detail by way of the embodiments which are only examples.

Furthermore, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices are disclosed and these devices can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps. These sensor control device embodiments can be used and can be capable of use to implement those steps performed by a sensor control device from any and all of the methods described herein.

As mentioned, a number of embodiments of systems, devices, and methods are described herein that provide for improvements to sensor insertion devices and sensor control devices for use with in vivo analyte monitoring systems. In particular, several embodiments of the present disclosure are designed to improve the longevity of certain components of a sensor applicator device and reduce their susceptibility to mechanical failure. Some embodiments, for example, include a pull-tab comprising an electrically insulative material coupled with one or more of: the sensor contacts, power supply, or another component of the sensor electronics. In certain embodiments, the pull-tab can be configured to prevent electrical coupling between the power supply and the sensor electronics of the sensor control device, thereby preserving the power supply of the sensor control device during storage or shipment. In another embodiment, a leaf spring retraction mechanism is implemented in a sensor applicator to reduce the number of potential sensor applicator device components susceptible to mechanical failure. In yet another embodiment, a plurality of magnetic elements is employed to retain a sensor control device in a sensor carrier of the applicator, and also reduces the number of sensor applicator device components susceptible to mechanical failure. Consequently, these embodiments can improve the longevity and functionality of sensor applicator devices and the sensor control devices, to name a few advantages.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Exemplary In Vivo Analyte Monitoring System

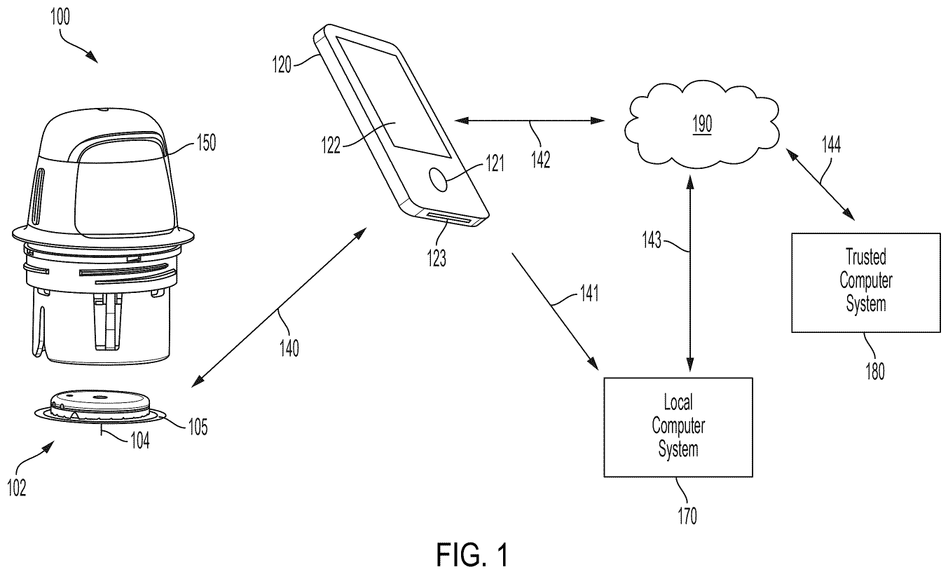

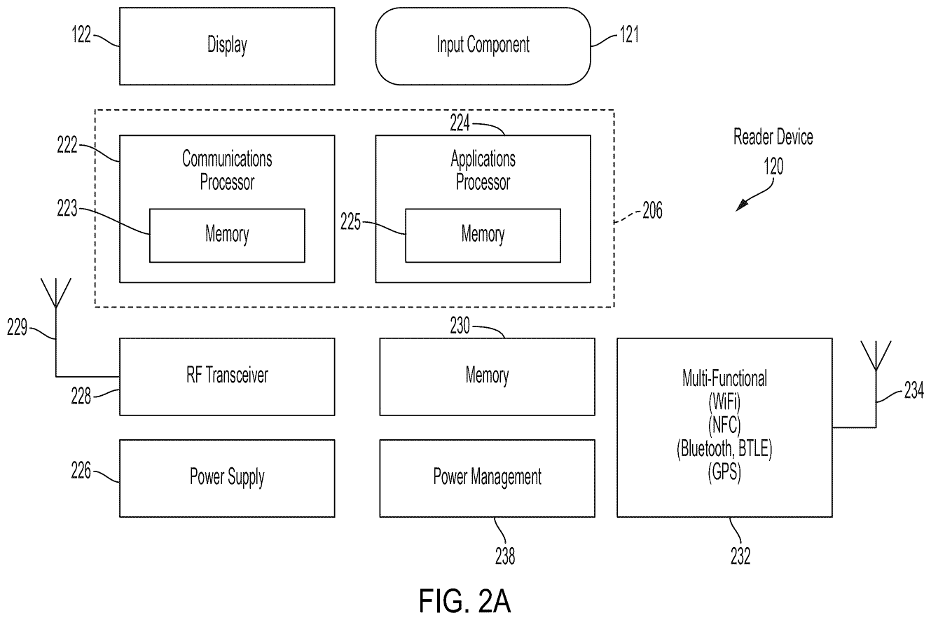

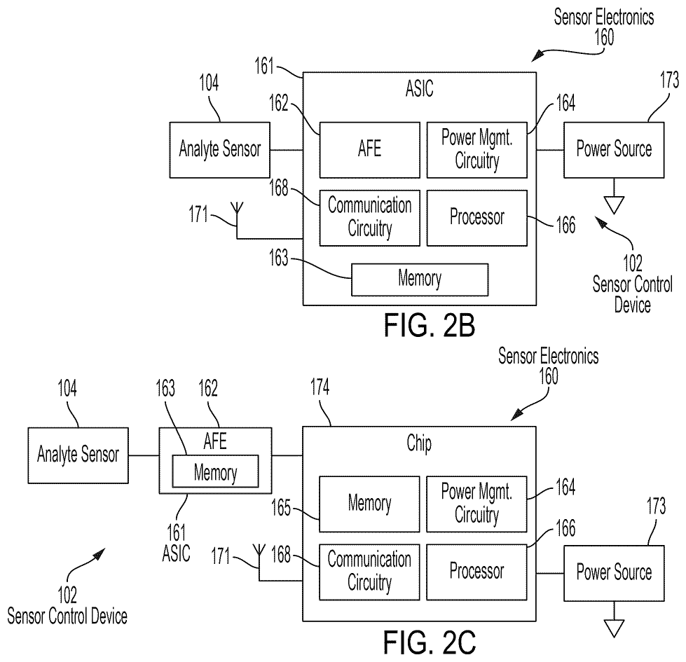

FIG. 1 is a conceptual diagram depicting an example embodiment of an analyte monitoring system 100 that includes a sensor applicator 150, a sensor control device 102, and a reader device 120. Here, sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can monitor applications installed in memory on reader device 120 using screen 122 and input 121 and the device battery can be recharged using power port 123. Reader device 120 can communicate with local computer system 170 via a communication path 141 using a wired or wireless technique. Local computer system 170 can include one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. Local computer system 170 can communicate via communications path 143 with a network 190 similar to how reader device 120 can communicate via a communications path 142 with network 190, by wired or wireless technique as described previously. Network 190 can be any of a number of networks, such as private networks and public networks, local area or wide area networks, and so forth. A trusted computer system 180 can include a server and can provide authentication services and secured data storage and can communicate via communications path 144 with network 190 by wired or wireless technique.

### Exemplary Reader Device

FIG. 2A is a block diagram depicting an example embodiment of a reader device configured as a smartphone. Here, reader device 120 can include a display 122, input component 121, and a processing core 206 including a communications processor 222 coupled with memory 223 and an applications processor 224 coupled with memory 225. Also included can be separate memory 230, RF transceiver 228 with antenna 229, and power supply 226 with power management module 238. Further included can be a multi-functional transceiver 232 which can communicate over Wi-Fi, NFC, Bluetooth, BTLE, and GPS with an antenna 234. As understood by one of skill in the art, these components are electrically and communicatively coupled in a manner to make a functional device.

### Exemplary Sensor Control Devices

FIGS. 2B and 2C are block diagrams depicting example embodiments of sensor control device 102 having analyte sensor 104 and sensor electronics 160 (including analyte monitoring circuitry) that can have the majority of the processing capability for rendering end-result data suitable for display to the user. In FIG. 2B, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 173, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to reader device 120 (not shown), for example, where minimal further processing is needed by the resident software application to display the data.

FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 includes memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

### Exemplary Assembly Processes for Sensor Control Devices

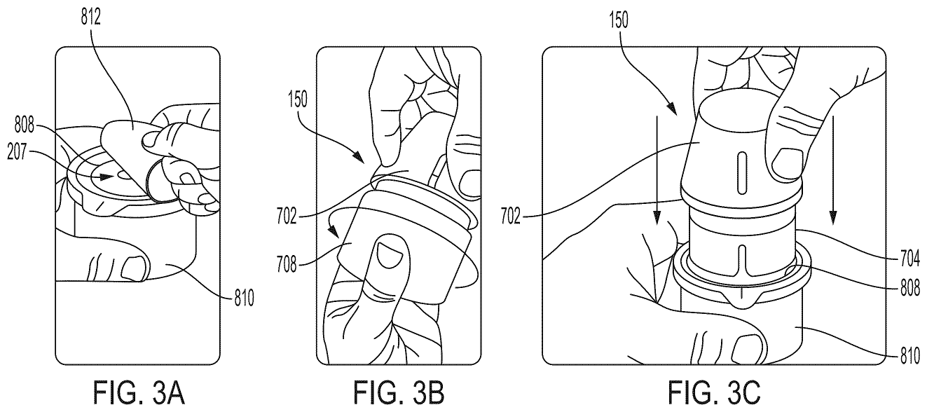

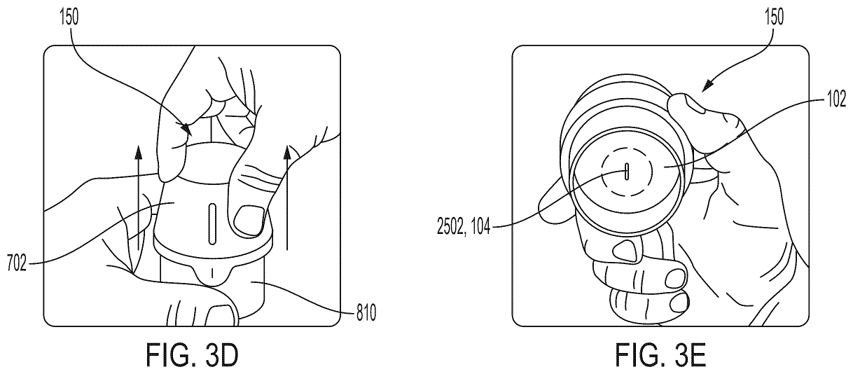

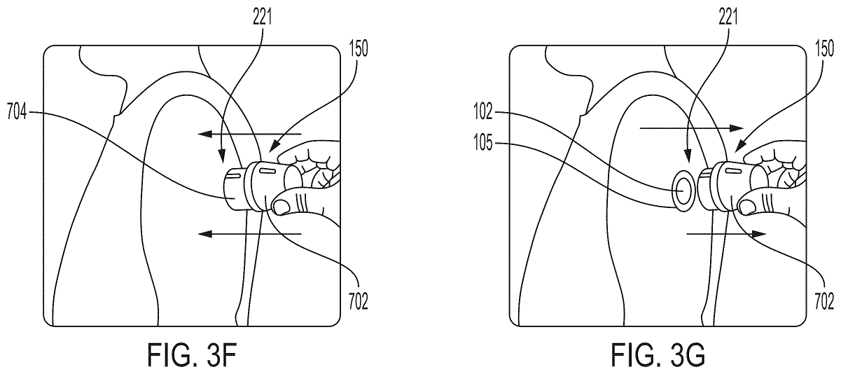

The components of sensor control device 102 can be acquired by a user in multiple packages requiring final assembly by the user before delivery to an appropriate user location. FIGS. 3A-3D depict an example embodiment of an assembly process for sensor control device 102 by a user, including preparation of separate components before coupling the components in order to ready the sensor for delivery. FIGS. 3E-3F depict an example embodiment of delivery of sensor control device 102 to an appropriate user location by selecting the appropriate delivery location and applying device 102 to the location.

FIG. 3A is a proximal perspective view depicting an example embodiment of a user preparing a container 810, configured here as a tray (although other packages can be used), for an assembly process. The user can accomplish this preparation by removing lid 812 from tray 810 to expose platform 808, for instance by peeling a non-adhered portion of lid 812 away from tray 810 such that adhered portions of lid 812 are removed. Removal of lid 812 can be appropriate in various embodiments so long as platform 808 is adequately exposed within tray 810. Lid 812 can then be placed aside.

FIG. 3B is a side view depicting an example embodiment of a user preparing an applicator device 150 for assembly. Applicator device 150 can be provided in a sterile package sealed by a cap 708. Preparation of applicator device 150 can include uncoupling housing 702 from cap 708 to expose sheath 704 (FIG. 3C). This can be accomplished by unscrewing (or otherwise uncoupling) cap 708 from housing 702. Cap 708 can then be placed aside.

FIG. 3C is a proximal perspective view depicting an example embodiment of a user inserting an applicator device 150 into a tray 810 during an assembly. Initially, the user can insert sheath 704 into platform 808 inside tray 810 after aligning housing orienting feature 1302 (or slot or recess) and tray orienting feature 924 (an abutment or detent). Inserting sheath 704 into platform 808 temporarily unlocks sheath 704 relative to housing 702 and also temporarily unlocks platform 808 relative to tray 810. At this stage, removal of applicator device 150 from tray 810 will result in the same state prior to initial insertion of applicator device 150 into tray 810 (i.e., the process can be reversed or aborted at this point and then repeated without consequence).

Sheath 704 can maintain position within platform 808 with respect to housing 702 while housing 702 is distally advanced, coupling with platform 808 to distally advance platform 808 with respect to tray 810. This step unlocks and collapses platform 808 within tray 810. Sheath 704 can contact and disengage locking features (not shown) within tray 810 that unlock sheath 704 with respect to housing 702 and prevent sheath 704 from moving (relatively) while housing 702 continues to distally advance platform 808. At the end of advancement of housing 702 and platform 808, sheath 704 is permanently unlocked relative to housing 702. A sharp and sensor (not shown) within tray 810 can be coupled with an electronics housing (not shown) within housing 702 at the end of the distal advancement of housing 702. Operation and interaction of the applicator device 150 and tray 810 are further described below.

FIG. 3D is a proximal perspective view depicting an example embodiment of a user removing an applicator device 150 from a tray 810 during an assembly. A user can remove applicator 150 from tray 810 by proximally advancing housing 702 with respect to tray 810 or other motions having the same end effect of uncoupling applicator 150 and tray 810. The applicator device 150 is removed with sensor control device 102 (not shown) fully assembled (sharp, sensor, electronics) therein and positioned for delivery.

FIG. 3E is a proximal perspective view depicting an example embodiment of a patient applying sensor control device 102 using applicator device 150 to a target area of skin, for instance, on an abdomen or other appropriate location. Advancing housing 702 distally collapses sheath 704 within housing 702 and applies the sensor to the target location such that an adhesive layer on the bottom side of sensor control device 102 adheres to the skin. The sharp is automatically retracted when housing 702 is fully advanced, while the sensor (not shown) is left in position to measure analyte levels.

FIG. 3F is a proximal perspective view depicting an example embodiment of a patient with sensor control device 102 in an applied position. The user can then remove applicator 150 from the application site.

System 100, described with respect to FIGS. 3A-3F and elsewhere herein, can provide a reduced or eliminated chance of accidental breakage, permanent deformation, or incorrect assembly of applicator components compared to prior art systems. Since applicator housing 702 directly engages platform 808 while sheath 704 unlocks, rather than indirect engagement via sheath 704, relative angularity between sheath 704 and housing 702 will not result in breakage or permanent deformation of the arms or other components. The potential for relatively high forces (such as in conventional devices) during assembly will be reduced, which in turn reduces the chance of unsuccessful user assembly.

### Exemplary Sensor Applicator Devices

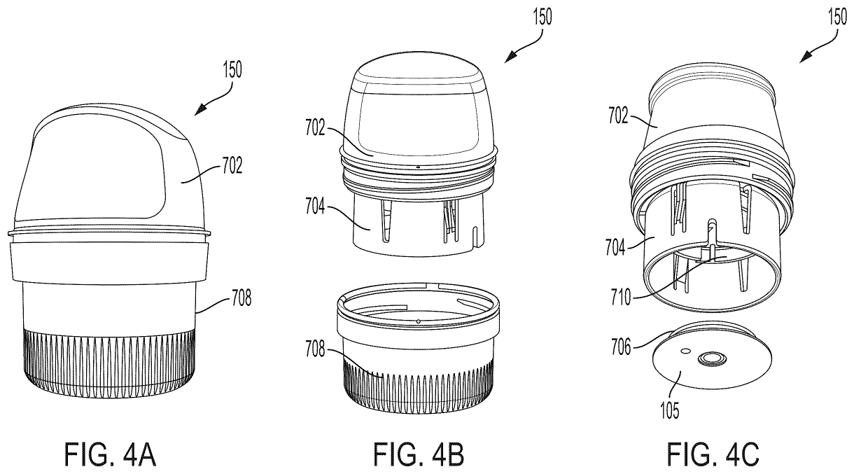

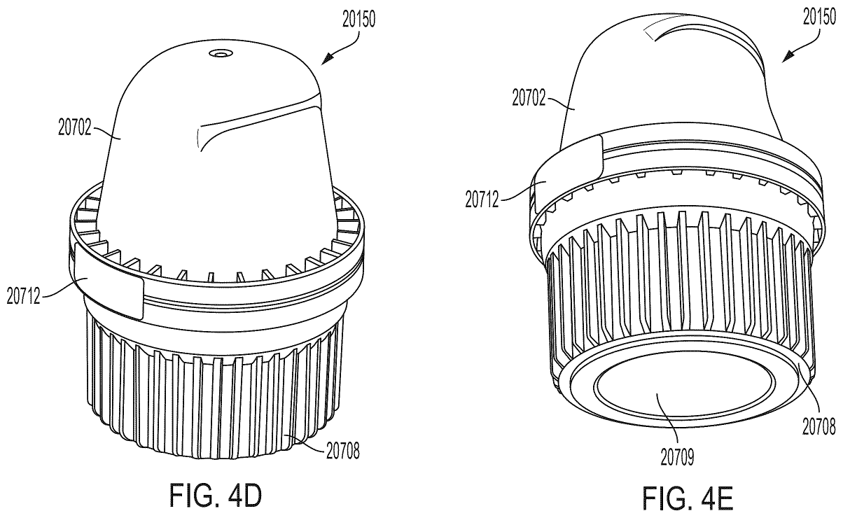

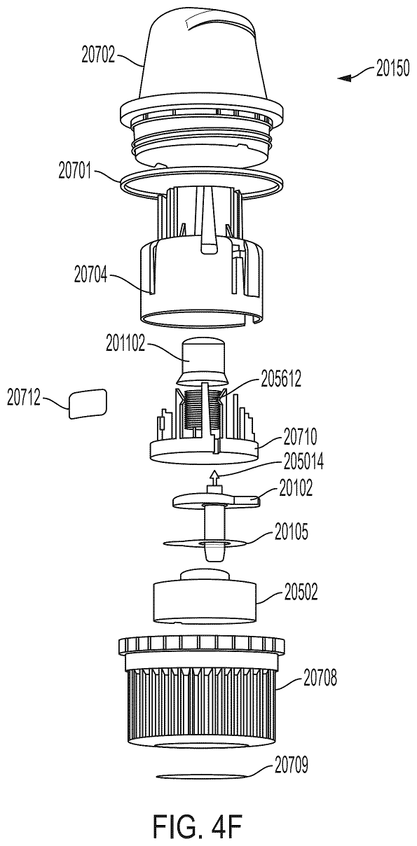

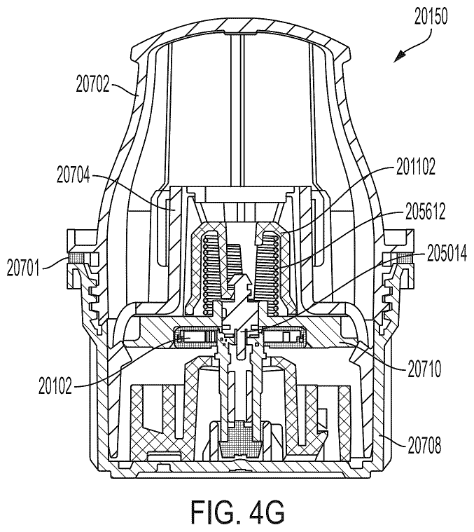

FIG. 4A is a side view depicting an example embodiment of an applicator device 150 coupled with screw cap 708. This is an example of how applicator 150 is shipped to and received by a user, prior to assembly by the user with a sensor. FIG. 4B is a side perspective view depicting applicator 150 and cap 708 after being decoupled. FIG. 4C is a perspective view depicting an example embodiment of a distal end of an applicator device 150 with electronics housing 706 and adhesive patch 105 removed from the position they would have retained within sensor carrier 710 of sheath 704, when cap 708 is in place.

Referring to FIG. 4D-G for purpose of illustration and not limitation, the applicator device 20150 can be provided to a user as a single integrated assembly. FIGS. 4D and 4E provide perspective top and bottom views, respectively, of the applicator device 20150, FIG. 4F provides an exploded view of the applicator device 20150 and FIG. 4G provides a side cut-away view. The perspective views illustrate how applicator 20150 is shipped to and received by a user. The exploded and cut-away views illustrate the components of the applicator device 20150. The applicator device 20150 can include a housing 20702, gasket 20701, sheath 20704, sharp carrier 201102, spring 205612, sensor carrier 20710 (also referred to as a “puck carrier”), sharp hub 205014, sensor control device (also referred to as a “puck”) 20102, adhesive patch 20105, desiccant 20502, cap 20708, serial label 20709, and tamper evidence feature 20712. As received by a user, only the housing 20702, cap 20708, tamper evidence feature 20712, and label 20709 are visible. The tamper evidence feature 20712 can be, for example, a sticker coupled to each of the housing 20702 and the cap 20708, and tamper evidence feature 20712 can be damaged, for example, irreparably, by uncoupling housing 20702 and cap 20708, thereby indicating to a user that the housing 20702 and cap 20708 have been previously uncoupled. These features are described in greater detail below.

Referring to FIG. 4G, in some embodiments, the interior of housing 20702 and cap 20708 can be lightly pressurized with an inert gas during assembly. According to one aspect of some embodiments, filling the interior of housing 20702 and cap 20708 with an inert, dry gas (e.g., nitrogen or argon) during assembly can be used either in place of, or in addition to, a desiccant. According to another aspect of some embodiments, lightly pressurizing the interior of housing 20702 and cap 20708 with an inert gas can also outwardly bias the flow of materials across the seal formed by housing 20702 and cap 20708, and can reduce the chance of ingress of unwanted contaminants. Moreover, as a further advantage of some embodiments, a slight release of gas pressure from the applicator when the user removes cap 20708 can be audible to indicate to the user that the seal has not been compromised during shipping and/or storage.

According to another aspect of some embodiments, an inert gas can be introduced into the interior of housing 20702 using a closed temperature-controlled system (not shown). First, one or more applicator devices 20150 can be placed into the closed system, while the closed system is at a first predetermined temperature. In some embodiments, the closed system can already be filled with the inert gas before the applicator devices 20150 are placed within. In other embodiments, the closed system can be filled with the inert gas after the applicator devices 20150 are placed within. The closed system is sealed such that the inert gas is prevented from escaping, while other external gases cannot enter. According to an aspect of some embodiments, when the closed system is maintained at the first predetermined temperature, applicator housing 20702 and cap 20708 form a seal, as described in the previous sections. Subsequently, the closed system is heated to a second predetermined temperature greater than the first predetermined temperature. According to an aspect of some embodiments, at the second predetermined temperature, for each of the one or more applicator devices 20150, the thermal expansion of cap 20708 can be different (e.g., greater or less) than the thermal expansion of the corresponding housing 20702, thereby unsealing each applicator device 20150. Consequently, the inert gas can diffuse into the interior of each housing 20702 while the applicator device 20150 is in an unsealed state. After a predetermined amount of time has elapsed that is sufficient to allow the inert gas to diffuse into each housing 20702, the temperature of the closed system can then be reduced to a lower temperature. In some embodiments, the lower temperature can be the first predetermined temperature. In other embodiments, the lower temperature can be a third predetermined temperature that is different from the first predetermined temperature, but is also lower than the second predetermined temperature. At the lower temperature, cap 20708 can contract such that the seal with the housing 20702 can be formed once again. Finally, the one or more applicators can be removed from the closed system.

### Exemplary Sensor Carriers

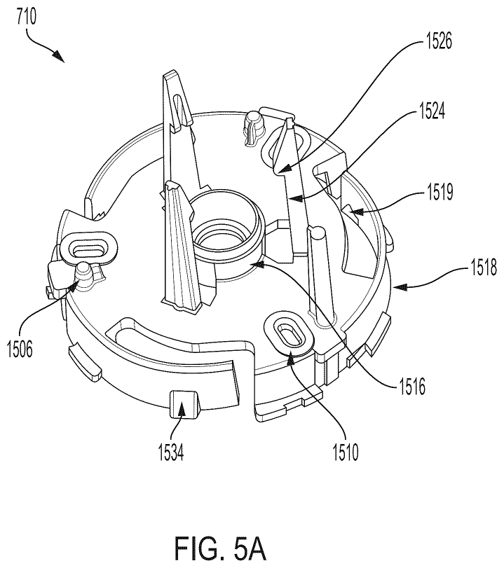

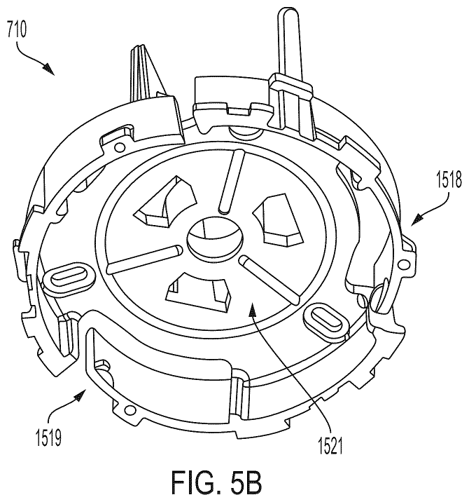

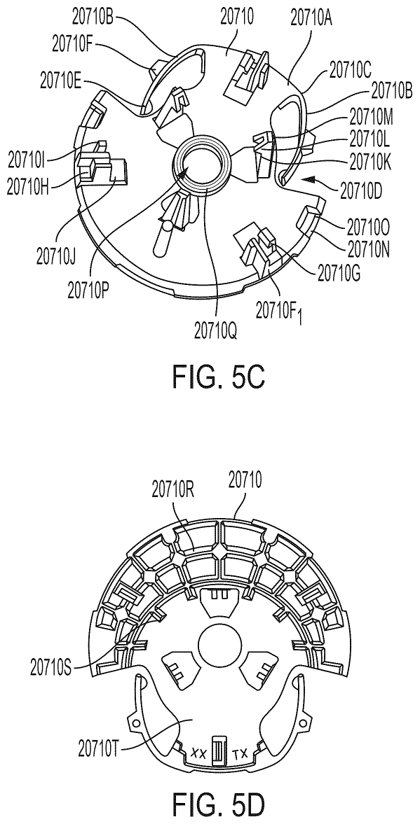

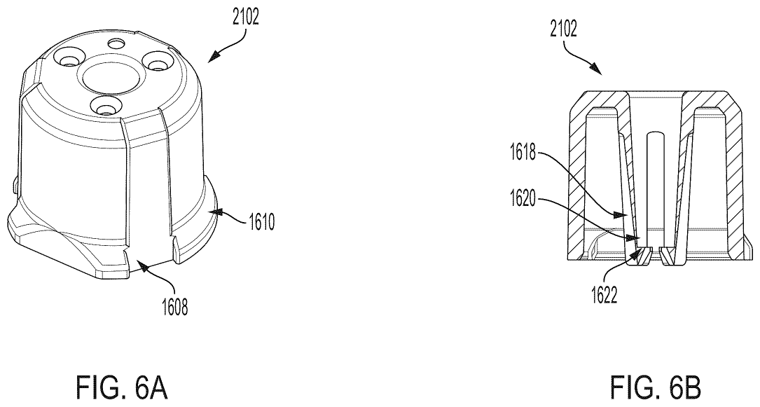

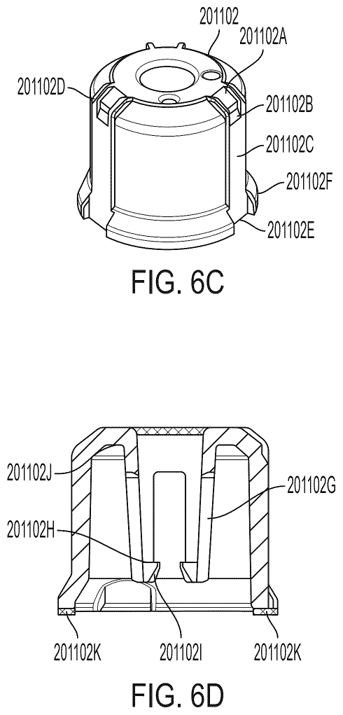

FIG. 5A is a proximal perspective view depicting an example embodiment of sensor carrier 710 that can retain sensor control device within applicator 150. It can also retain sharp carrier 2102 with sharp module 2500. In this example embodiment, sensor carrier 710 generally has a hollow round flat cylindrical shape, and can include one or more deflectable sharp carrier lock arms 1524 (e.g., three) extending proximally from a proximal surface surrounding a centrally located spring alignment ridge 1516 for maintaining alignment of spring 1104. Each lock arm 1524 has a detent or retention feature 1526 located at or near its proximal end. Shock lock 1534 can be a tab located on an outer circumference of sensor carrier 710 extending outward and can lock sensor carrier 710 for added safety prior to firing. Rotation limiter 1506 can be a proximally extending relatively short protrusion on a proximal surface of sensor carrier 710 which limits rotation of carrier 710. Sharp carrier lock arms 1524 can interface with sharp carrier 2102 as described with reference to FIGS. 6A-6E below.

FIG. 5B is a distal perspective view of sensor carrier 710. Here, one or more sensor electronics retention spring arms 1518 (e.g., three) are normally biased towards the position shown and include a detent 1519 that can pass over the distal surface of electronics housing 706 of device 102 when housed within recess or cavity 1521. In certain embodiments, after sensor control device 102 has been adhered to the skin with applicator 150, the user pulls applicator 150 in a proximal direction, i.e., away from the skin. The adhesive force retains sensor control device 102 on the skin and overcomes the lateral force applied by spring arms 1518. As a result, spring arms 1518 deflect radially outwardly and disengage detents 1519 from sensor control device 102 thereby releasing sensor control device 102 from applicator 150.

Referring to FIGS. 5C and 5D, for purpose of illustration and not limitation, an exemplary sensor carrier 20710 is provided. Sensor carrier 20710 can include one or more of the features described herein with regard to sensor carriers, wherein similar features can operate as described herein. For example, sensor carrier 20710 can include a base 20710A and first and second retention arms 20710B. Each retention arm 20710B can include a first end portion 20710C coupled to the base 20710A and a free end portion 20710D. For example, each retention arm 20710B can be coupled to the base 20710A at a first half of the base 20710A and the free end portion 20710D can extend toward a second half of the base 20710A. Each retention arm 20710B can include a sensor retention feature 20710E disposed on an inner surface of the sensor retention arm 20710B. The sensor retention feature 20710E can be disposed on the free end portion 20710D. The sensor retention feature 20710E can be configured to retain the sensor control device 20102 within the housing 20702. The retention feature 20710E can include a conical surface and angular parting line, which can allow for release of the sensor control device 20102 upon delivery. Each retention arm 20710 can include a lock interface 20710F disposed on an outer surface of the retention arm 20710B. The lock interface 20710F can engage rib 20704U on the sheath 20704. As described hereinabove, the rib 20704U can prevent the sensor retention arm 20710B from flexing outwardly, for example, during a shock event, and therefore can keep retention feature 20710E engaged with the sensor control device 20102, and thereby prevent movement of the sensor control device 20102 during a shock event.

In some embodiments, sensor control device (e.g., 102 or 20102) can be retained in sensor carrier (e.g., 710 or 20710) by one or more magnets (not shown) disposed on a sharp carrier. According to one aspect of some embodiments, and as further described below with respect to FIGS. 6A-6D, one or more magnets disposed in the sharp carrier can be configured to attract one or more ferromagnetic components disposed in sensor control device (e.g., 102 or 20102) and, consequently, retain sensor control device (e.g., 102 or 20102) in sensor carrier (e.g., 710 or 20710). In some embodiments, the one or more ferromagnetic components can be disposed in a housing of the sensor control device (e.g., 102 or 20102). In some embodiments, the one or more magnets can be implemented either in addition to, or in place of, the one or more sensor electronics retention spring arms 1518 and corresponding detents 1519 of sensor carrier 710 (FIGS. 5A-5B), or retention arms 20710B with corresponding sensor retention features 20710E of sensor carrier 20710 (FIGS. 5C-5D). In certain embodiments, it may be preferable to implement the one or more magnets without spring arms 1518, detents 1519, retention arms 201710B, and sensor retention features 20710E because such structural features can be subject to adverse conditions during storage or use that can cause, for example, material creep over time.

In other embodiments, sensor control device (e.g., 102 or 20102) can be retained in sensor carrier (e.g., 710 or 20710) by one or more magnets disposed in the sensor carrier itself. According to one aspect of some embodiments, one advantage of locating the one or more magnets in the sensor carrier (e.g., 710 or 20710) is the proximity between the one or more magnets and the sensor control device (e.g., 102 or 20102). As a result, less magnetic force may be required because, in some embodiments, the one or more magnets in the sensor carrier can be configured to directly engage at least a portion of the sensor control device (e.g., a top portion). Furthermore, according to another aspect of some embodiments, the adhesive patch can be configured such that the adhesive property is greater than the magnetic force between the one or more magnets and the sensor control device. Accordingly, after the sensor control unit reaches the distal position and the adhesive patch is coupled with the skin, the sensor control unit can disengage from the sensor carrier when the user pulls the applicator device away from the skin.

Referring back to FIGS. 5C and 5D, sensor carrier 20710 can include a plurality of housing attachment features 20710F1. In some embodiments, for example, sensor carrier 20710 can include three housing attachment features 20710F1. In other embodiments, sensor carrier 20710 can include two, four, five, six, or more housing attachment features 20710F1. The housing attachment features 20710F1 can be equally spaced on the sensor carrier 20710 and can extend upwardly from a top surface of the sensor carrier 20710. Each sensor housing attachment feature 20710F1 can include a housing snap 20710G, housing locator feature 20710H, biasing feature 20710I, and housing stop 20710J. The housing locator feature 20710H can axially locate the sensor carrier 20710 relative the housing 20702 when the two are to be coupled together. The housing snap 20710G can engage the sensor carrier attach slots 20702K on the housing 20702 to couple the sensor carrier 20710 to the housing 20702. The biasing feature 20710I can engage sensor carrier biasing feature 20702M on housing 20702 configured to remove slop between the sensor carrier 20710 and the housing 20702.

Sensor carrier 20710 can further include a plurality of sharp carrier lock arms 20710K, for example three sharp carrier lock arms 20710K. The sharp carrier lock arms 20710K can be equally spaced on the sensor carrier 20710 and can extend upwardly form a top surface of the sensor carrier 20710. Each sharp carrier lock arm 20710K can include a sharp carrier retention feature 20710L and a rib 20710M. Rib 20710M can engage an inner surface of the sheath 20704, which can urge the sharp carrier lock arm 20710K inwardly and cause sharp carrier retention feature 20710L to retain sharp carrier 201102, as described in greater detail below. The carrier retention feature 20710L can have a triangle shape when viewed in side view and a “U” shape when viewed in top view.

In accordance with the disclosed subject matter, the sensor carrier 20710 can include a plurality of lock ledges 20710N configured to engage lock arm interface 20704M of the sheath 20704 as described herein above. For example, the sensor carrier 20710 can include two lock ledges 20710N. Sensor carrier 20710 can include recesses 207100 disposed proximate each lock ledge 20710N and configured to receive the lock arm interface 20704M during firing, to prevent the lock arm 20704J from engaging with housing 20702 during firing. Sensor carrier 20710 can include a hole 20710P extending through a middle of the base 20710A. The hole 20710P can guide and limit movement of sharp hub 205014 during insertion. Additionally, or alternatively, sensor carrier 20710 can include spring locator 20710Q.

A bottom surface of the sensor carrier 20710 can include stiffening ribs 20710R and sensor locator ribs 20710S, which can limit planar motion of the sensor control device 20102 relative the sensor carrier 20710. The bottom surface of the sensor carrier 20710 can include a sensor support surface 20710T configure to support the sensor control device 20102.

### Exemplary Sharp Carriers

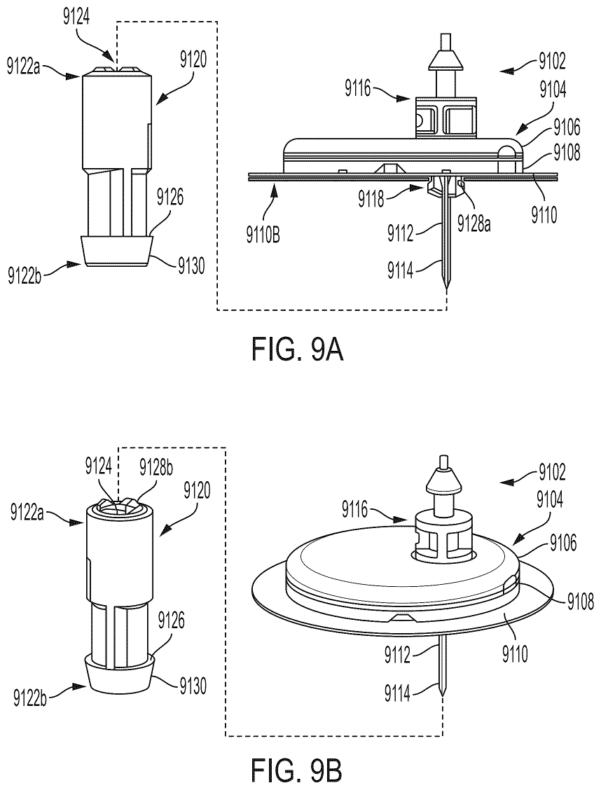

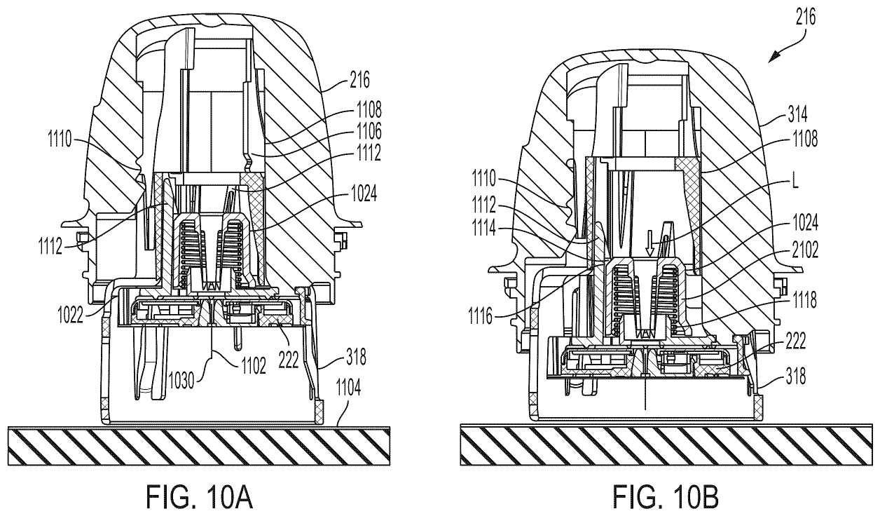

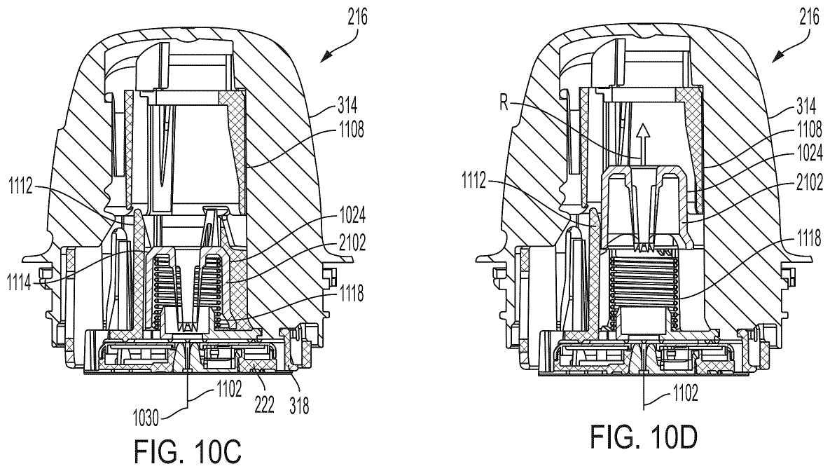

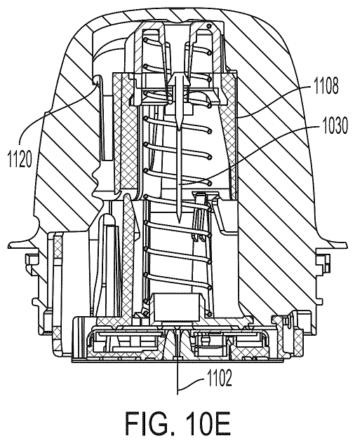

FIGS. 6A and 6B are a proximal perspective view and a side cross-sectional view, respectively, depicting an example embodiment of sharp carrier 2102. Sharp carrier 2102 can grasp and retain sharp module 2500 within applicator 150. It can also automatically retract as a result of one or more springs changing from a preloaded, compressed state to an expanded state during an insertion process, as described with respect to FIGS. 10A-10E. Near a distal end of sharp carrier 2102 can be anti-rotation slots 1608 which prevent sharp carrier 2102 from rotating when located within a central area of sharp carrier lock arms 1524 (as shown in FIG. 9A). Anti-rotation slots 1608 can be located between sections of sharp carrier base chamfer 1610, which can ensure full retraction of sharp carrier 2102 through sheath 704 upon retraction of sharp carrier 2102 at the end of the deployment procedure.

As shown in FIG. 6B, sharp retention arms 1618 can be located in an interior of sharp carrier 2102 about a central axis and can include a sharp retention clip 1620 at a distal end of each arm 1618. Sharp retention clip 1620 can have a proximal surface which can be nearly perpendicular to the central axis and can abut a distally facing surface of sharp hub 2516.

Referring to FIGS. 6C and 6D, for purpose of illustration and not limitation, an exemplary sharp carrier 201102 is provided. Sharp carrier 201102 can include one or more features described herein with regard to sharp carriers, wherein similar features can operate as describe herein. For example, sharp carrier 201102 can include a series of features for engaging with the three sharp carrier lock arms 20710K of the sensor carrier 20710. The features can include a pre-partial-retraction retention face 201102A and a post-partial-retraction retention face 201102B. The pre-partial retraction retention face 201102A can engage the sharp carrier retention feature 20710L prior to partial retraction, for example, during shipping and storage. Post-partial-retraction retention face 201102B can engage the sharp carrier retention feature 20710L after partial retraction. For example, as the sheath 20704 initially move proximally relative to the sensor carrier 20710, the rib 20710M of the retention arm 20710L can engage slot 20704Q of sheath 20704, which can allow the retention arm 20710L to move radially outward and allow sharp carrier retention feature 20710L to clear the pre-partial retraction retention face 201102A and engage the post-partial retraction retention face 201102B. A height between the end of the pre-partial-retraction face 201102A and the start of the post-partial-retraction face 201102B can be the distance of the partial retraction. A running face 201102C can be disposed below the post-partial-retraction retention face 201102B and can slide against the retention arm 20710L as the sharp carrier 201102 is retracted. Alignment walls 201102D can help to keep the sharp carrier 201102 aligned with the sensor carrier 20704 during partial retraction. Sharp carrier 201102 can include a chamfer 201102F, which can include anti-rotation slots 201102E to engage the retention arms 20710L on the sensor carrier 20710.

Internally, sharp carrier 201102 can include sharp retention arms 201102G including lead-in face 2011021 and sharp hub contact face 201102H. The retention arms 201102G can receive and hold sharp hub 205014. Spring stop 201102J can engage retraction spring 205612.

Referring to FIG. 6D, according to some embodiments, sharp carrier 201102 can also comprise one or more magnets 201102K for retaining a sensor control device in the sensor carrier (e.g., 710 or 20710 of FIGS. 5A-5D). By way of illustration, sharp carrier 201102 can comprise one or more magnets 201102K disposed in or on a distal-facing surface of sharp carrier 201102. According to one aspect of these embodiments, the one or more magnets 201102K are configured to attract one or more ferromagnetic components disposed in the sensor control device, which can cause the sensor control device to be retained in the sensor carrier when sharp carrier 201102 and sensor carrier are proximate to each other. More specifically, when sharp carrier 201102K and sensor carrier are coupled, as depicted in FIGS. 10A-10C, the one or more magnets 201102K are configured to generate a magnetic field having sufficient strength to exert a “pulling” force upon the ferromagnetic components disposed in the sensor control device in a proximal direction, such that the sensor control device is retained in the sensor carrier.

According to another aspect of the embodiments, as shown in FIG. 10E, the expansion of the return spring during the retraction of the sharp causes sharp carrier 201102 to separate from the sensor carrier and to be displaced in a proximal direction. As sharp carrier 201102 moves further away from the sensor carrier, the one or more magnets 201102K no longer exert a sufficient magnetic force to retain the sensor control device in the sensor carrier. Subsequently, the sensor control device can disengage from the sensor carrier.

According to some embodiments, the one or more magnets 201102K can be embedded in a distal end of the sharp carrier 201102 such that the distal-facing surface is flush against the sensor carrier. In some embodiments, the one or more magnets 201102K can comprise either a single magnetic element or a plurality of discrete magnetic elements. For example, in some embodiments, the one or more magnets 201102K can comprise a single magnetic element having an annular geometry. In other embodiments, the one or more magnets 201102K can comprise two, three, four, five or more discrete magnetic elements disposed upon the distal facing surface of sharp carrier 201102. In still other embodiments, at least a portion of the distal end of sharp carrier 201102 itself can be constructed from a magnetic material. Those of skill in the art will appreciate that other configurations and geometries for implementing the one or more magnets for retaining a sensor control device in the sensor carrier are possible and fully within the scope of the present disclosure.

### Exemplary Sensor and Connector Assemblies

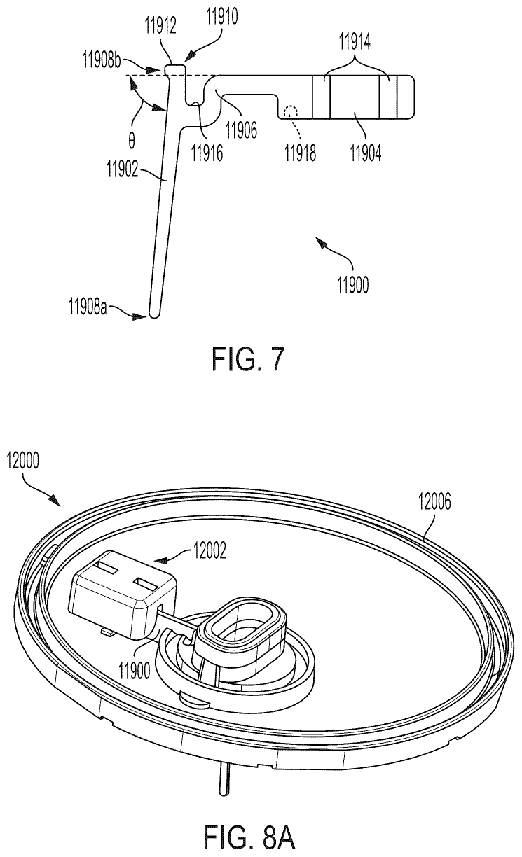

FIG. 7 is a side view of an example sensor 11900, according to one or more embodiments of the disclosure. The sensor 11900 may be similar in some respects to any of the sensors described herein and, therefore, may be used in an analyte monitoring system to detect specific analyte concentrations. As illustrated, the sensor 11900 includes a tail 11902, a flag 11904, and a neck 11906 that interconnects the tail 11902 and the flag 11904. The tail 11902 includes an enzyme or other chemistry or biologic and, in some embodiments, a membrane may cover the chemistry. In use, the tail 11902 is transcutaneously received beneath a user's skin, and the chemistry included thereon helps facilitate analyte monitoring in the presence of bodily fluids.

The tail 11902 may be received within a hollow or recessed portion of a sharp (not shown) to at least partially circumscribe the tail 11902 of the sensor 11900. As illustrated, the tail 11902 may extend at an angle Q offset from horizontal. In some embodiments, the angle Q may be about 85°. Accordingly, in contrast to other sensor tails, the tail 11902 may not extend perpendicularly from the flag 11904, but instead at an angle offset from perpendicular. This may prove advantageous in helping maintain the tail 11902 within the recessed portion of the sharp.

The tail 11902 includes a first or bottom end 11908a and a second or top end 11908b opposite the bottom end 11908a. A tower 11910 may be provided at or near the top end 11908b and may extend vertically upward from the location where the neck 11906 interconnects the tail 11902 to the flag 11904. During operation, if the sharp moves laterally, the tower 11910 will help pivot the tail 11902 toward the sharp and otherwise stay within the recessed portion of the sharp. Moreover, in some embodiments, the tower 11910 may provide or otherwise define a protrusion 11912 that extends laterally therefrom. When the sensor 11900 is mated with the sharp and the tail 11902 extends within the recessed portion of the sharp, the protrusion 11912 may engage the inner surface of the recessed portion. In operation, the protrusion 11912 may help keep the tail 11902 within the recessed portion.

The flag 11904 may comprise a generally planar surface having one or more sensor contacts 11914 arranged thereon. The sensor contact(s) 11914 may be configured to align with a corresponding number of compliant carbon impregnated polymer modules encapsulated within a connector.

In some embodiments, as illustrated, the neck 11906 may provide or otherwise define a dip or bend 11916 extending between the flag 11904 and the tail 11902. The bend 11916 may prove advantageous in adding flexibility to the sensor 11900 and helping prevent bending of the neck 11906.

In some embodiments, a notch 11918 (shown in dashed lines) may optionally be defined in the flag near the neck 11906. The notch 11918 may add flexibility and tolerance to the sensor 11900 as the sensor 11900 is mounted to the mount. More specifically, the notch 11918 may help take up interference forces that may occur as the sensor 11900 is mounted within the mount.

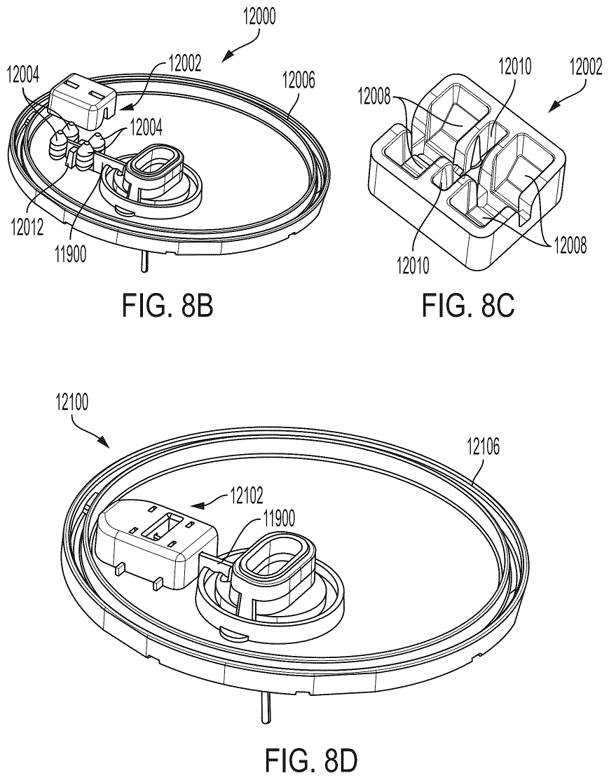

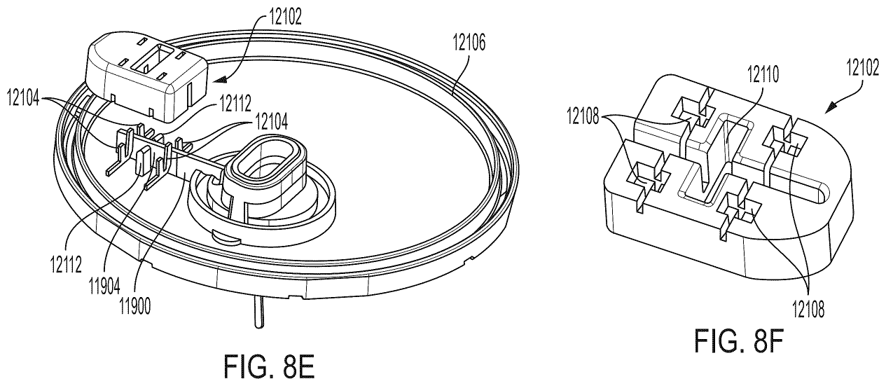

FIGS. 8A and 8B are isometric and partially exploded isometric views of an example connector assembly 12000, according to one or more embodiments. As illustrated, the connector assembly 12000 may include a connector 12002, and FIG. 8C is an isometric bottom view of the connector 12002. The connector 12002 may comprise an injection molded part used to help secure one or more compliant carbon impregnated polymer modules 12004 (four shown in FIG. 8B) to a mount 12006. More specifically, the connector 12002 may help secure the modules 12004 in place adjacent the sensor 11900 and in contact with the sensor contacts 11914 (FIG. 7C) provided on the flag 11904 (FIG. 7C). The modules 12004 may be made of a conductive material to provide conductive communication between the sensor 11900 and corresponding circuitry contacts (not shown) provided within the mount 12006.

As best seen in FIG. 8C, the connector 12002 may define pockets 12008 sized to receive the modules 12004. Moreover, in some embodiments, the connector 12002 may further define one or more depressions 12010 configured to mate with one or more corresponding flanges 12012 (FIG. 8B) on the mount 12006. Mating the depressions 12010 with the flanges 12012 may secure the connector 12002 to the mount 12006 via an interference fit or the like. In other embodiments, the connector 12002 may be secured to the mount 12006 using an adhesive or via sonic welding.

FIGS. 8D and 8E are isometric and partially exploded isometric views of another example connector assembly 12100, according to one or more embodiments. As illustrated, the connector assembly 12100 may include a connector 12102, and FIG. 8F is an isometric bottom view of the connector 12102. The connector 12102 may comprise an injection molded part used to help keep one or more compliant metal contacts 12104 (four shown in FIG. 8E) secured against the sensor 11900 on a mount 12106. More specifically, the connector 12102 may help secure the contacts 12104 in place adjacent the sensor 11900 and in contact with the sensor contacts 11914 (FIG. 7C) provided on the flag 11904. The contacts 12104 may be made of a stamped conductive material that provides conductive communication between the sensor 11900 and corresponding circuitry contacts (not shown) provided within the mount 12106. In some embodiments, for example, the contacts 12104 may be soldered to a PCB (not shown) arranged within the mount 12106.

As best seen in FIG. 8F, the connector 12102 may define pockets 12108 sized to receive the contacts 12104. Moreover, in some embodiments, the connector 12102 may further define one or more depressions 12110 configured to mate with one or more corresponding flanges 12112 on the mount 12006. Mating the depressions 12110 with the flanges 12112 may help secure the connector 12102 to the mount 12106 via an interference fit or the like. In other embodiments, the connector 12102 may be secured to the mount 12106 using an adhesive or via sonic welding.

In some embodiments, connector assembly (e.g., 12000 or 12100) can include a pull-tab (not shown) constructed from one or more electrically insulative materials configured to prolong the life of the battery and/or prevent current leakage during storage. According to some embodiments, for example, a first portion of the pull-tab can be removably engaged with sensor 11900 to prevent electrical coupling between sensor contacts 11914, on the one hand, and either of modules 12004 of FIG. 8B or contacts 12104 in FIG. 8E, on the other hand. Furthermore, in some embodiments, a second portion of the pull-tab can be coupled with the sharp or sharp carrier such that the pull-tab is disengaged from sensor 11900 when the applicator is actuated. In other embodiments, the second portion of the pull-tab can be coupled with the sharp or sharp carrier such that the pull-tab is disengaged from sensor 11900 during or after the retraction of the sharp.

According to other embodiments, a first portion of a pull-tab (not shown) can be removably engaged with the power supply (e.g., battery) to prevent electrical coupling between the power supply and the rest of the sensor electronics (e.g., PCB). In said embodiments, the second portion of the pull-tab can be coupled with the sharp or sharp carrier such that the pull-tab is disengaged from the power supply either when the applicator is actuated, or during (or after) the retraction of the sharp. In still other embodiments, a first portion of a pull-tab (not shown) can be removably engaged with any componentry of sensor electronics in the sensor control unit that would otherwise create a closed circuit with the power supply. Those of skill in the art will recognize that other configurations to preserve battery life and prevent current leakage during storage are possible, and are fully within the scope of the present disclosure.

### Example Embodiments of Sensor Control Devices

FIGS. 9A and 9B are side and isometric views, respectively, of an example sensor control device 9102, according to one or more embodiments of the present disclosure. The sensor control device 9102 may be similar in some respects to the sensor control device 102 of FIG. 1 and therefore may be best understood with reference thereto. Moreover, the sensor control device 9102 may replace the sensor control device 102 of FIG. 1 and, therefore, may be used in conjunction with the sensor applicator 102 of FIG. 1, which may deliver the sensor control device 9102 to a target monitoring location on a user's skin.

As illustrated, the sensor control device 9102 includes an electronics housing 9104, which may be generally disc-shaped and have a circular cross-section. In other embodiments, however, the electronics housing 9104 may exhibit other cross-sectional shapes, such as ovoid, oval, or polygonal, without departing from the scope of the disclosure. The electronics housing 9104 includes a shell 9106 and a mount 9108 that is mateable with the shell 9106. The shell 9106 may be secured to the mount 9108 via a variety of ways, such as a snap fit engagement, an interference fit, sonic welding, laser welding, one or more mechanical fasteners (e.g., screws), a gasket, an adhesive, or any combination thereof. In some cases, the shell 9106 may be secured to the mount 9108 such that a sealed interface is generated therebetween. An adhesive patch 9110 may be positioned on and otherwise attached to the underside of the mount 9108. According to an aspect of the embodiments, the adhesive patch 9110 (shown in FIG. 9A in non-hatched shading) may be configured to secure and maintain the sensor control device 9102 in position on the user's skin during operation.

The sensor control device 9102 may further include a sensor 9112 and a sharp 9114 used to help deliver the sensor 9112 transcutaneously under a user's skin during application of the sensor control device 9102. Corresponding portions of the sensor 9112 and the sharp 9114 extend distally from the bottom of the electronics housing 9104 (e.g., the mount 9108). A sharp hub 9116 may be overmolded onto the sharp 9114 and configured to secure and carry the sharp 9114. As best seen in FIG. 9A, the sharp hub 9116 may include or otherwise define a mating member 9118. In assembling the sharp 9114 to the sensor control device 9102, the sharp 9114 may be advanced axially through the electronics housing 9104 until the sharp hub 9116 engages an upper surface of the electronics housing 9104 or an internal component thereof and the mating member 9118 extends distally from the bottom of the mount 9108. As described herein below, in at least one embodiment, the sharp hub 9116 may sealingly engage an upper portion of a seal overmolded onto the mount 9108. As the sharp 9114 penetrates the electronics housing 9104, the exposed portion of the sensor 9112 may be received within a hollow or recessed (arcuate) portion of the sharp 9114. The remaining portion of the sensor 9112 is arranged within the interior of the electronics housing 9104.

The sensor control device 9102 may further include a sensor cap 9120, shown detached from the electronics housing 9104 in FIGS. 9A-9B. The sensor cap 9120 may help provide a sealed barrier that surrounds and protects exposed portions of the sensor 9112 and the sharp 9114. As illustrated, the sensor cap 9120 may comprise a generally cylindrical body having a first end 9122a and a second end 9122b opposite the first end 9122a. The first end 9122a may be open to provide access into an inner chamber 9124 defined within the body. In contrast, the second end 9122b may be closed and may provide or otherwise define an engagement feature 9126. As described in more detail below, the engagement feature 9126 may help mate the sensor cap 9120 to an applicator cap of a sensor applicator (e.g., the sensor applicator 102 of FIG. 1), and may help remove the sensor cap 9120 from the sensor control device 9102 upon removing the sensor cap from the sensor applicator.

The sensor cap 9120 may be removably coupled to the electronics housing 9104 at or near the bottom of the mount 9108. More specifically, the sensor cap 9120 may be removably coupled to the mating member 9118, which extends distally from the bottom of the mount 9108. In at least one embodiment, for example, the mating member 9118 may define a set of external threads 9128a (FIG. 9A) mateable with a set of internal threads 9128b (FIG. 9B) defined within the inner chamber 9124 of the sensor cap 9120. In some embodiments, the external and internal threads 9128a,b may comprise a flat thread design (e.g., lack of helical curvature), but may alternatively comprise a helical threaded engagement. Accordingly, in at least one embodiment, the sensor cap 9120 may be threadably coupled to the sensor control device 9102 at the mating member 9118 of the sharp hub 9116. In other embodiments, the sensor cap 9120 may be removably coupled to the mating member 9118 via other types of engagements including, but not limited to, an interference or friction fit, or a frangible member or substance (e.g., wax, an adhesive, etc.) that may be broken with minimal separation force (e.g., axial or rotational force).

In some embodiments, the sensor cap 9120 may comprise a monolithic (singular) structure extending between the first and second ends 9122a,b. In other embodiments, however, the sensor cap 9120 may comprise two or more component parts. In the illustrated embodiment, for example, the body of the sensor cap 9120 may include a desiccant cap 9130 arranged at the second end 9122b. The desiccant cap 9130 may house or comprise a desiccant to help maintain preferred humidity levels within the inner chamber 9124. Moreover, the desiccant cap 9130 may also define or otherwise provide the engagement feature 9126 of the sensor cap 9120. In at least one embodiment, the desiccant cap 9130 may comprise an elastomeric plug inserted into the bottom end of the sensor cap 9120.

In some embodiments, sensor control device 9102 can also include an adhesive liner 9110B (shown in FIG. 9A in hatched shading) coupled with a bottom surface of adhesive patch 9110. Under certain conditions within the housing and cap of the applicator, chemical interactions between the adhesive patch 9110, trapped atmosphere, desiccant, and outgassing of materials can cause the adhesive to degrade during storage or shipment of the applicator. Applying liner 9110B can mitigate degradation of the adhesive of adhesive patch 9110. According to another aspect of the embodiments, liner 9110B can also be operatively coupled with sensor cap 9120 such that removal of sensor cap 9120 also causes removal of liner 9110B.

### Exemplary Firing Mechanism of One-Piece and Two-Piece Applicators

FIGS. 10A-10E illustrate example details of embodiments of the internal device mechanics of “firing” the applicator 216 to apply sensor control device 222 to a user and including retracting sharp 1030 safely back into used applicator 216. All together, these drawings represent an example sequence of driving sharp 1030 (supporting a sensor coupled to sensor control device 222) into the skin of a user, withdrawing the sharp while leaving the sensor behind in operative contact with interstitial fluid of the user, and adhering the sensor control device to the skin of the user with an adhesive. Modification of such activity for use with the alternative applicator assembly embodiments and components can be appreciated in reference to the same by those with skill in the art. Moreover, applicator 216 may be a sensor applicator having one-piece architecture or a two-piece architecture as disclosed herein.

Turning now to FIG. 10A, a sensor 1102 is supported within sharp 1030, just above the skin 1104 of the user. Rails 1106 (optionally three of them) of an upper guide section 1108 may be provided to control applicator 216 motion relative to sheath 318. The sheath 318 is held by detent features 1110 within the applicator 216 such that appropriate downward force along the longitudinal axis of the applicator 216 will cause the resistance provided by the detent features 1110 to be overcome so that sharp 1030 and sensor control device 222 can translate along the longitudinal axis into (and onto) skin 1104 of the user. In addition, catch arms 1112 of sensor carrier 1022 engage the sharp retraction assembly 1024 to maintain the sharp 1030 in a position relative to the sensor control device 222.

In FIG. 10B, user force is applied to overcome or override detent features 1110 and sheath 318 collapses into housing 314 driving the sensor control device 222 (with associated parts) to translate down as indicated by the arrow L along the longitudinal axis. An inner diameter of the upper guide section 1108 of the sheath 318 constrains the position of carrier arms 1112 through the full stroke of the sensor/sharp insertion process. The retention of the stop surfaces 1114 of carrier arms 1112 against the complimentary faces 1116 of the sharp retraction assembly 1024 maintains the position of the members with return spring 1118 fully energized.

In FIG. 10C, sensor 1102 and sharp 1030 have reached full insertion depth. In so doing, the carrier arms 1112 clear the upper guide section 1108 inner diameter. Then, the compressed force of the coil return spring 1118 drives angled stop surfaces 1114 radially outward, releasing force to drive the sharp carrier 2102 of the sharp retraction assembly 1024 to pull the (slotted or otherwise configured) sharp 1030 out of the user and off of the sensor 1102 as indicated by the arrow R in FIG. 10D.

With the sharp 1030 fully retracted as shown in FIG. 10E, the upper guide section 1108 of the sheath 318 is set with a final locking feature 1120. Subsequently, the spent applicator assembly 216 is removed from the insertion site, leaving behind the sensor control device 222, and with the sharp 1030 secured safely inside the applicator assembly 216. The spent applicator assembly 216 is now ready for disposal.

Operation of the applicator 216 when applying the sensor control device 222 is designed to provide the user with a sensation that both the insertion and retraction of the sharp 1030 is performed automatically by the internal mechanisms of the applicator 216. In other words, the present invention avoids the user experiencing the sensation that he is manually driving the sharp 1030 into his skin. Thus, once the user applies sufficient force to overcome the resistance from the detent features of the applicator 216, the resulting actions of the applicator 216 are perceived to be an automated response to the applicator being “triggered.” The user does not perceive that he is supplying additional force to drive the sharp 1030 to pierce his skin despite that all the driving force is provided by the user and no additional biasing/driving means are used to insert the sharp 1030. As detailed above in FIG. 10C, the retraction of the sharp 1030 is automated by the coil return spring 1118 of the applicator 216.

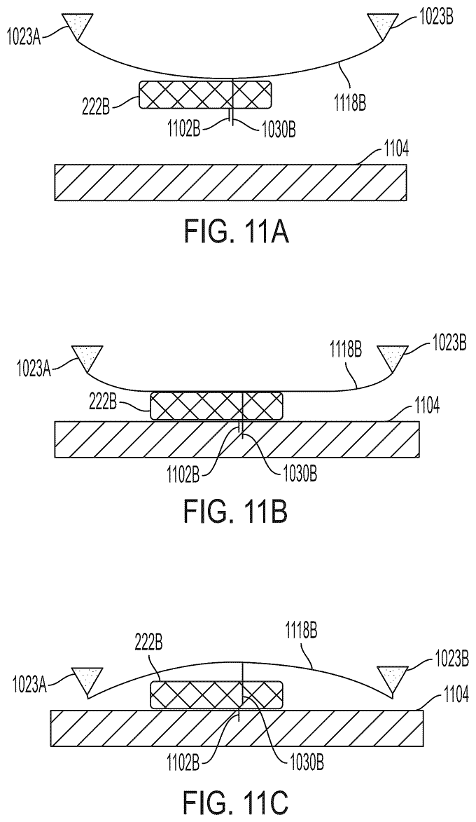

FIGS. 11A-11C depict an alternative embodiment of a spring-biased retraction mechanism for implementation in a sensor applicator device. According to one aspect of some embodiments, in order to reduce the number of components in a sensor applicator (and reduce the number of potential mechanical failures), a leaf spring 1118B can be employed in a sensor applicator, such as applicator 216 of FIGS. 10A-10E, in place of a coiled return spring 1118.

Referring first to FIG. 11A, a partial cross-sectional side view of certain sensor applicator components, according to some embodiments, is shown in a pre-firing stage (similar to FIG. 10A). In particular, FIG. 11A depicts leaf spring 1118B coupled with sharp 1030B, wherein sharp 1030B is in a spaced relation with the skin surface 1104. As further shown in FIG. 11A, leaf spring 1118B is depicted in a first state in which a distal facing surface of leaf spring 1118B is in a convex configuration relative to skin surface 1104.

According to some embodiments, sharp 1030B can be coupled with a center portion of leaf spring 1118B by an interference fit, sonic welding, laser welding, one or more mechanical fasteners (e.g., screws), a gasket, an adhesive, or any combination thereof. In some embodiments, leaf spring 1118B can be constructed from the same material (e.g., stainless steel) as sharp 1030B. In other embodiments, leaf spring 1118B can be constructed from a first material (e.g., stainless steel) having a first stiffness, and sharp 1030B can be constructed from a second material (e.g., plastic) having a second stiffness different than the first stiffness. According to another aspect of some embodiments, like the embodiment depicted in FIG. 10A, sharp 1030B can extend through sensor control device 222B, and a portion of glucose sensor 1102B can be coupled with, or partially disposed within, a distal portion of sharp 1030B.

According to another aspect of some embodiments, a plurality of engagement features 1023A, 1023B are configured to affix leaf spring 1118B with either sensor carrier (not shown) or a sharp retraction assembly (not shown), such that the downward movement of the housing, sensor carrier and sharp retraction assembly will also cause at least the edge portions of leaf spring 1118B to move in the distal direction.

FIG. 11B is another partial cross-sectional side view of the aforementioned sensor applicator components, according to some embodiments, wherein the applicator is depicted in an insertion stage (similar to FIG. 10C). In particular, FIG. 11B shows sharp 1030B after it has pierced skin surface 1104, and sensor 1102B has reached a predetermined insertion depth. According to an aspect of some embodiments, at the insertion stage, the adhesive pad (not shown) on the bottom surface of sensor control device 222B is adhered to skin surface 1104.

As can be further seen in FIG. 11B, leaf spring 1118B is depicted in a second state in which the surface of leaf spring 1118B has been transformed from a convex configuration (relative to skin surface 1104) to a substantially planar configuration, as the housing, sensor carrier, and sharp retraction assembly (not shown) of the applicator continue to advance in a distal direction. In some embodiments, the substantially planar surface of leaf spring 1118B can also be configured to apply a force in a distal direction against either the sensor control device 222B or the sensor carrier (not shown).

FIG. 11C is another partial cross-section side view of the aforementioned applicator components, according to some embodiments, wherein the applicator is shown in a retraction stage (similar to FIGS. 10D and 10E). In particular, as the housing (not shown) of the applicator is further displaced in the distal direction, leaf spring 1118B is depicted in a third state in which the surface of leaf spring 1118B has reached or exceeded a deformation threshold level such that the leaf spring 1118B “snaps” into a concave configuration relative skin surface 1104. As a result of the concave configuration, according to an aspect of some embodiments, sharp 1030B is retracted in a proximal direction away from skin surface 1104, while leaving behind sensor 1102B under skin surface 1104. In some embodiments, the concave configuration can also cause leaf spring 1118B to disengage from sensor control device 222B.

Subsequently, according to some embodiments, the applicator can be removed from the insertion site, leaving behind the sensor control device 222B, and with the sharp 1030B secured safely inside the applicator assembly. The applicator assembly can now be disposed of.

With respect to any of the applicator embodiments described herein, as well as any of the components thereof, including but not limited to the sharp, sharp module and sensor module embodiments, those of skill in the art will understand that said embodiments can be dimensioned and configured for use with sensors configured to sense an analyte level in a bodily fluid in the epidermis, dermis, or subcutaneous tissue of a subject. In some embodiments, for example, sharps and distal portions of analyte sensors disclosed herein can both be dimensioned and configured to be positioned at a particular end-depth (i.e., the furthest point of penetration in a tissue or layer of the subject's body, e.g., in the epidermis, dermis, or subcutaneous tissue). With respect to some applicator embodiments, those of skill in the art will appreciate that certain embodiments of sharps can be dimensioned and configured to be positioned at a different end-depth in the subject's body relative to the final end-depth of the analyte sensor. In some embodiments, for example, a sharp can be positioned at a first end-depth in the subject's epidermis prior to retraction, while a distal portion of an analyte sensor can be positioned at a second end-depth in the subject's dermis. In other embodiments, a sharp can be positioned at a first end-depth in the subject's dermis prior to retraction, while a distal portion of an analyte sensor can be positioned at a second end-depth in the subject's subcutaneous tissue. In still other embodiments, a sharp can be positioned at a first end-depth prior to retraction and the analyte sensor can be positioned at a second end-depth, wherein the first end-depth and second end-depths are both in the same layer or tissue of the subject's body.

Additionally, with respect to any of the applicator embodiments described herein, those of skill in the art will understand that an analyte sensor, as well as one or more structural components coupled thereto, including but not limited to one or more spring-mechanisms, can be disposed within the applicator in an off-center position relative to one or more axes of the applicator. In some applicator embodiments, for example, an analyte sensor and a spring mechanism can be disposed in a first off-center position relative to an axis of the applicator on a first side of the applicator, and the sensor electronics can be disposed in a second off-center position relative to the axis of the applicator on a second side of the applicator. In other applicator embodiments, the analyte sensor, spring mechanism, and sensor electronics can be disposed in an off-center position relative to an axis of the applicator on the same side. Those of skill in the art will appreciate that other permutations and configurations in which any or all of the analyte sensor, spring mechanism, sensor electronics, and other components of the applicator are disposed in a centered or off-centered position relative to one or more axes of the applicator are possible and fully within the scope of the present disclosure.

A number of deflectable structures are described herein, including but not limited to deflectable detent snaps, deflectable locking arms, sharp carrier lock arms, sharp retention arms, and module snaps. These deflectable structures are composed of a resilient material such as plastic or metal (or others) and operate in a manner well known to those of ordinary skill in the art. The deflectable structures each has a resting state or position that the resilient material is biased towards. If a force is applied that causes the structure to deflect or move from this resting state or position, then the bias of the resilient material will cause the structure to return to the resting state or position once the force is removed (or lessened). In many instances these structures are configured as arms with detents, or snaps, but other structures or configurations can be used that retain the same characteristics of deflectability and ability to return to a resting position, including but not limited to a leg, a clip, a catch, an abutment on a deflectable member, and the like.

Additional details of suitable devices, systems, methods, components and the operation thereof along with related features are set forth in International Publication No. WO2018/136898 to Rao et. al., International Publication No. WO2019/236850 to Thomas et. al., International Publication No. WO2019/236859 to Thomas et. al., International Publication No. WO2019/236876 to Thomas et. al., and U.S. Patent Publication No. 2020/0196919, filed Jun. 6, 2019, each of which is incorporated by reference in its entirety herein. Further details regarding embodiments of applicators, their components, and variants thereof, are described in U.S. Patent Publication Nos. 2012/0197222, 2013/0150691, 2016/0128615, 2016/0331283, 2018/0235520, 2019/0298240, and 2020/0397356 all of which are incorporated by reference herein in their entireties and for all purposes. Further details regarding embodiments of sharp modules, sharps, their components, and variants thereof, are described in U.S. Patent Publication No. 2014/0171771, which is incorporated by reference herein in its entirety and for all purposes.

Exemplary embodiments and features are set out in the following numbered clauses:
1. A sensor applicator assembly comprising:
   a housing configured to move between a first position and a second position;
   a sheath slidably coupled with the housing; and
   a sensor carrier coupled with the housing;
   an applicator cap threadably coupled with the housing,
   wherein the applicator cap and the housing define an interior space, and wherein the interior space comprises a lightly pressurized inert gas.
2. The sensor applicator assembly of clause 1, wherein the housing comprises an applicator cap sealing lip configured to interface with the applicator cap.
3. The sensor applicator assembly of clause 2, wherein the applicator cap includes a seal interface configured to receive the applicator cap sealing lip of the housing.
4. The sensor applicator assembly of clause 3, wherein the seal interface and the applicator cap sealing lip are configured to form a seal between the housing and the applicator cap.
5. The sensor applicator assembly of clause 4, wherein the seal further comprises a gasket.
6. The sensor applicator assembly of clause 4 or 5, wherein the lightly pressurized inert gas creates an outwardly biased flow across the seal.
7. The sensor applicator assembly of any of clauses 1 to 6, wherein the lightly pressurized gas comprises nitrogen.
8. The sensor applicator assembly of any of clauses 1 to 7, wherein the applicator cap is configured to retain a desiccant.
9. The sensor applicator assembly of any of clauses 1 to 8, wherein the applicator cap does not include a desiccant.
10. The sensor applicator assembly of any of clauses 1 to 9, wherein the interior space comprises a first pressure, and wherein an exterior space external to the sensor applicator assembly comprises a second pressure that is less than the first pressure.
11. A sensor applicator assembly comprising:
   a housing configured to move between a first position and a second position;
   a sheath slidably coupled with the housing;
   a sharp carrier comprising one or more magnets;
   a sensor control device comprising an analyte sensor, sensor electronics, and one or more ferromagnetic components; and
   a sensor carrier configured to retain the sensor control device,
   wherein the one or more magnets are configured to exert a magnetic force upon the ferromagnetic components in a proximate direction, such that the sensor control device is retained in the sensor carrier when the housing is in the first position.
12. The sensor applicator assembly of clause 11, wherein the one or more ferromagnetic components are disposed in the sensor control device.
13. The sensor applicator assembly of clause 11 or 12, wherein the one or more ferromagnetic components are embedded in a housing of the sensor control device.
14. The sensor applicator assembly of any of clauses 11 to 13, wherein the sensor carrier is configured to retain the sensor control device by magnetic force only.
15. The sensor applicator assembly of any of clauses 11 to 14, wherein the one or more magnets are disposed on a distal-facing surface of the sharp carrier.
16. The sensor applicator assembly of any of clauses 11 to 15, wherein the one or more magnets are embedded in a distal end of the sharp carrier.
17. The sensor applicator assembly of any of clauses 11 to 16, wherein the one or more magnets comprise a single magnetic element.
18. The sensor applicator assembly of clause 17, wherein the single magnetic element comprises an annular geometry.
19. The sensor applicator assembly of clauses 11 to 18, wherein at least a portion of a distal end of the sharp carrier comprises a magnetic material.
20. The sensor applicator assembly of clauses 11 to 16, wherein the one or more magnets comprises two magnetic elements disposed upon a distal facing surface of the sharp carrier.
21. The sensor applicator assembly of clauses 11 to 16, wherein the one or more magnets comprises three magnetic elements disposed upon a distal facing surface of the sharp carrier.
22. The sensor applicator assembly of clauses 11 to 21, further comprising a return spring.
23. The sensor applicator assembly of clause 22, wherein the return spring is configured to expand and move the sharp carrier in a proximate direction after the housing has reached the second position.
24. The sensor applicator assembly of clause 23, wherein the one or more magnets are configured such that the magnetic force exerted upon the ferromagnetic components is not sufficient to retain the sensor control device in the sensor carrier after the sharp carrier has moved in the proximate direction.
25. The sensor applicator assembly of clause 24, wherein the sensor control device is configured to disengage from the sensor carrier after the sharp carrier has moved in the proximate direction.
26. A sensor applicator assembly comprising:
   a housing configured to move between a first position and a second position;
   a sheath slidably coupled with the housing;
   a sharp carrier coupled with a sharp;
   a sensor control device comprising:
   an analyte sensor comprising a plurality of sensor contacts,
   a connector assembly comprising one or more of a plurality of sensor modules or a plurality of connector contacts, the connector assembly configured to couple with the analyte sensor, and
   a power supply; and
   a sensor carrier configured to retain the sensor control device,
   wherein the connector assembly further comprises a pull-tab comprising an electrically insulative material.
27. The sensor applicator assembly of clause 26, wherein the pull-tab comprises a first portion removably engaged with the plurality of sensor contacts.
28. The sensor applicator assembly of clause 27, wherein the first portion of the pull-tab is configured to prevent electrical coupling between the sensor contacts and the plurality of sensor modules.
29. The sensor applicator assembly of clause 27, wherein the first portion of the pull-tab is configured to prevent electrical coupling between the sensor contacts and the plurality of connector contacts.
30. The sensor applicator assembly of any of clauses 27 to 29, wherein the pull-tab comprises a second portion coupled with the sharp or the sharp carrier.
31. The sensor applicator assembly of clause 30, wherein the pull-tab is configured to disengage from the plurality of sensor contacts by movement of the sharp or the sharp carrier when the sensor applicator assembly is actuated.
32. The sensor applicator assembly of clause 30, wherein the pull-tab is configured to disengage from the plurality of sensor contacts when the sharp or the sharp carrier is retracted within the sensor applicator assembly.
33. A sensor applicator assembly comprising:
   a housing configured to move between a first position and a second position;
   a sheath slidably coupled with the housing;
   a sharp carrier coupled with a sharp;
   a sensor control device comprising:
   an analyte sensor comprising a plurality of sensor contacts,
   a connector assembly configured to couple with the analyte sensor, and
   a power supply; and
   a sensor carrier configured to retain the sensor control device,
   wherein the sensor control device further comprises a pull-tab coupled with the power supply, wherein the pull-tab comprises an electrically insulative material.
34. The sensor applicator assembly of clause 33, wherein the power supply is a coin cell battery.
35. The sensor applicator assembly of clause 33 or 34, wherein the pull-tab comprises a first portion removably engaged with the power supply.
36. The sensor applicator assembly of clause 35, wherein the first portion of the pull-tab is configured to prevent electrical coupling between the power supply and sensor electronics of the sensor control device.
37. The sensor applicator assembly of clause 35 or 36, wherein the pull-tab comprises a second portion coupled with the sharp or the sharp carrier.
38. The sensor applicator assembly of clause 37, wherein the pull-tab is configured to disengage from the power supply by movement of the sharp or the sharp carrier when the sensor applicator assembly is actuated.
39. The sensor applicator assembly of clause 37, wherein the pull-tab is configured to disengage from the power supply when the sharp or the sharp carrier is retracted within the sensor applicator assembly.
40. A sensor applicator assembly comprising:
   a housing configured to move between a first position and a second position;
   a sheath slidably coupled with the housing;
   a sharp carrier coupled with a sharp;
   a sensor control device comprising:
   an electronics housing,
   sensor electronics disposed within the electronics housing,
   an analyte sensor coupled with the sensor electronics,
   an adhesive patch disposed on a bottom surface of the electronics housing, and
   an adhesive liner coupled with a bottom surface of the adhesive patch; and
   a sensor carrier configured to retain the sensor control device; and
   a sensor cap removably coupled with the sensor control device.
41. The sensor applicator assembly of clause 40, wherein adhesive liner is operatively coupled with the sensor cap such that removal of the sensor cap causes removal of the adhesive liner.
42. A sensor applicator assembly of clause 41, further comprising an applicator cap threadably coupled with the housing, wherein the applicator cap is configured to remove the sensor cap from the sensor applicator assembly when the applicator cap is decoupled from the housing.
43. The applicator assembly of clause 41 or 42,
   wherein the sensor control device comprises a first aperture on a top surface of the electronics housing,
   wherein the sensor control device comprises a second aperture on the bottom surface of the electronics housing,
   wherein the adhesive patch comprises a third aperture,
   wherein the adhesive liner comprises a fourth aperture, and
   wherein the sharp extends through the first, second, third, and fourth apertures when the housing is in the first position.
44. A sensor applicator assembly comprising:
   a housing configured to move between a first position and a second position;
   a sheath slidably coupled with the housing, wherein a distal end of the sheath is configured to be placed against a skin surface;
   a leaf spring coupled with a sharp;
   a sensor control device comprising an analyte sensor coupled with sensor electronics; and
   a sensor carrier configured to retain the sensor control device when the housing is in the first position,
   wherein the sharp is configured to position at least a portion of the analyte sensor under the skin surface when the housing is moved to the second position, and
   wherein the leaf spring is configured to retract the sharp into the sensor applicator assembly after the housing is moved to the second position.
45. The sensor applicator assembly of clause 44, wherein the leaf spring comprises a convex configuration relative to the skin surface when the housing is in the first position.
46. The sensor applicator assembly of clause 44 or 45, wherein the leaf spring is configured to deform while the housing is moving between the first position and the second position.
47. The sensor applicator assembly of any of clauses 44 to 46, wherein the leaf spring comprises a substantially planar configuration relative to the skin surface before the sharp is retracted into the sensor applicator assembly.
48. The sensor applicator assembly of any of clauses 44 to 47,
   wherein the sensor control device is configured to be adhere to the skin surface when the housing is moved to the second position, and
   wherein the leaf spring comprises a substantially planar configuration relative to the skin surface when the sensor control device is adhered to the skin surface.
49. The sensor applicator assembly of any of clauses 44 to 48, wherein the leaf spring comprises a concave configuration relative to the skin surface after the sharp is retracted into the sensor applicator assembly.
50. The sensor applicator assembly of any of clauses 44 to 49, wherein the leaf spring is coupled to the sharp by an interference fit.
51. The sensor applicator assembly of any of clauses 44 to 49, wherein the leaf spring is coupled to the sharp by sonic welding.
52. The sensor applicator assembly of any of clauses 44 to 49, wherein the leaf spring is coupled to the sharp by laser welding.
53. The sensor applicator assembly of any of clauses 44 to 49, wherein the leaf spring is coupled to the sharp by one or more mechanical fasteners.
54. The sensor applicator assembly of any of clauses 44 to 53, wherein the leaf spring comprises a first material, and the sharp comprises a second material different from the first material.
55. The sensor applicator assembly of any of clauses 44 to 53, wherein the leaf spring and the sharp comprise a stainless steel material.
56. The sensor applicator assembly of any of clauses 44 to 55, wherein the leaf spring comprises a first stiffness, and wherein the sharp comprises a second stiffness different than the first stiffness.
57. The sensor applicator assembly of any of clauses 44 to 56, further comprising a plurality of engagement features configured to affix the leaf spring with the sensor carrier.
58. The sensor applicator assembly of any of clauses 44 to 56, further comprising a plurality of engagement features configured to affix the leaf spring with the sharp carrier.
59. The sensor applicator assembly of any of clauses 44 to 58, wherein the leaf spring is further configured to apply a force in a distal direction against the sensor control device.
60. The sensor applicator assembly of any of clauses 44 to 59, wherein the leaf spring is further configured to apply a force in a distal direction against the sensor carrier.
61. A method for introducing an inert gas into a sensor applicator assembly comprising a housing and a cap, the method comprising:
   placing the sensor applicator assembly into a closed system while the closed system is at a first predetermined temperature, wherein at the first predetermined temperature, the housing and the cap form a seal;
   heating the closed system from the first predetermined temperature to a second predetermined temperature, wherein at the second predetermined temperature, a thermal expansion of the cap causes the cap to unseal from the housing;
   diffusing the inert gas into an interior of the sensor applicator assembly; and
   cooling the closed system from the second predetermined temperature to a third predetermined temperature, wherein at the third predetermined temperature, contraction of the cap causes the cap to form the seal with the housing.
62. The method of clause 61, wherein the inert gas is argon.
63. The method of clause 61 or 62, wherein the first predetermined temperature is equal to the third predetermined temperature.
64. The method of any of clauses 61 to 63, wherein diffusing the inert gas into the interior of the sensor applicator assembly includes maintaining the closed system at the second predetermined temperature for a predetermined period of time.
65. The method of any of clauses 61 to 64, further comprising introducing the inert gas into the closed system before placing the sensor applicator assembly therein.
66. The method of any of clauses 61 to 64, further comprising introducing the inert gas into the closed system after placing the sensor applicator assembly therein.
67. A sensor applicator assembly comprising:
   a housing configured to move between a first position and a second position;
   a sheath slidably coupled with the housing;
   a sensor control device comprising an analyte sensor, sensor electronics, and one or more ferromagnetic components; and
   a sensor carrier comprising one or more magnets, the sensor carrier configured to retain the sensor control device,
   wherein the one or more magnets are configured to exert a magnetic force upon the ferromagnetic components in a proximate direction, such that the sensor control device is retained in the sensor carrier when the housing is in the first position.
68. The sensor applicator assembly of clause 67, wherein the one or more ferromagnetic components are disposed in the sensor control device.
69. The sensor applicator assembly of clause 67 or 68, wherein the sensor control device further comprises an adhesive patch.
70. The sensor applicator assembly of clause 69, wherein the adhesive patch, when coupled with a skin surface, creates an adhesive force greater than the magnetic force.
71. The sensor applicator assembly of clause 70, wherein the adhesive patch is configured such that the adhesive force causes the sensor control device to disengage from the sensor carrier when the sensor control device is adhered to the skin.
72. A sensor applicator assembly comprising:
   a housing comprising an interior;
   a sensor carrier configured to retain a sensor control device and to move between a first position and a second position within the interior of the housing, wherein the sensor carrier comprises a magnet;
   the sensor control device comprising a glucose sensor coupled with sensor electronics, the
   wherein the sensor control device is disposed within the interior of the housing when the sensor carrier is in the first position.
73. The sensor applicator assembly of claim 72, further comprising a sharp carrier, a sharp, and a return spring.
74. The sensor applicator assembly of claim 73, wherein the return spring is configured to expand and move the sharp carrier in a proximate direction.
75. The sensor control assembly of claim 72, wherein the magnet engages at least a portion of the sensor control device.
76. The sensor control assembly of claim 72, wherein the sensor control device comprises a material responsive to a magnetic field created by the magnet of the sensor carrier.
77. The sensor control assembly of claim 72, wherein the sensor control device further comprises an adhesive patch disposed on a bottom surface of the sensor control device.

The description encompasses and expressly envisages methods that are non-surgical, non-invasive methods implemented outside the body. The methods are typically implemented by a user who is not required to be a medical professional.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. Thus, the foregoing description of specific embodiments of the disclosed subject matter has been presented for purposes of illustration and description. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents. Furthermore, any features, functions, steps, or elements of the embodiments may be recited in or added to the claims, as well as negative limitations that define the inventive scope of the claims by features, functions, steps, or elements that are not within that scope.

1. (canceled)

2. An assembly for insertion of a glucose sensor in a subject's body, the assembly comprising:
(1) an applicator, comprising:
an applicator housing defining an interior;
a sheath slidably coupled with the applicator housing and comprising a distal end configured for placement on a subject's skin;
a sharp carrier assembly coupled with a sharp;
a sensor carrier comprising a cavity formed by a plurality of walls, the plurality of walls including a proximal wall, a side wall, and a magnetic component, and wherein the cavity is configured to retain a sensor control device;
a spring comprising a distal end in contact with the sensor carrier; and
a cap configured to couple with a distal part of the applicator housing;
(2) the sensor control device configured to be worn on the subject's body, the sensor control device comprising:
an adhesive patch disposed on a bottom surface of the sensor control device and configured to adhere the sensor control device to the subject's skin;
the glucose sensor comprising:
a proximal portion configured to electrically couple with electronics;
a distal portion having a glucose-responsive enzyme, wherein the distal portion is configured to be positioned in a bodily fluid of the subject's body and to sense glucose in the subject's bodily fluid in a subcutaneous layer; and
the electronics comprising one or more processors, a memory, and communication circuitry configured to wirelessly transmit data according to a Bluetooth or a Bluetooth Low Energy protocol,
wherein the sharp carrier assembly, the sharp, the spring, the sensor carrier, the magnetic component, and the sensor control device are configured to advance a distance within the interior of the applicator housing, relative to the sheath, and in a linear direction from a proximal position to a distal position,
wherein, in the proximal position, a distal end of the glucose sensor is received in a portion of the sharp,
wherein the magnetic component is configured to exert a magnetic force upon one or more components of the sensor control device when the sensor carrier and the sensor control device are in the proximal position, and
wherein, in the distal position, the sensor control device is configured to adhere to the subject's skin via the adhesive patch, and disengage from the sensor carrier when the user pulls the applicator away from the skin.

3. The assembly of claim 2, wherein the spring is configured to retract the sharp carrier assembly and the sharp in a proximal direction into the applicator after the sharp carrier assembly, the sharp, the spring, the sensor carrier, the magnetic component, and the sensor control device reach the distal position.

4. The assembly of claim 3, wherein the proximal portion of the glucose sensor comprises one or more electrical contacts.

5. The assembly of claim 4, wherein the glucose sensor further comprises a bent portion between the proximal portion of the glucose sensor and the distal portion of the glucose sensor.

6. The assembly of claim 5, wherein the proximal portion of the glucose sensor is perpendicular to the distal portion of the glucose sensor.

7. The assembly of claim 5, wherein the applicator further comprises a gasket configured to create a seal between the applicator housing and the cap.

8. The assembly of claim 5, wherein the distal end of the glucose sensor and a distal portion of the sharp extend in a distal direction from the bottom surface of the sensor control device when the sharp carrier assembly, the sharp, the spring, the sensor carrier, the magnetic component, and the sensor control device are in the proximal position.

9. The assembly of claim 8, wherein the sensor control device further comprises a first aperture on a top surface of the housing of the sensor control device,
wherein the sensor control device further comprises a second aperture on the bottom surface of the sensor control device, and
wherein the sharp is configured to extend through the first aperture and the second aperture when the sharp carrier assembly, the sharp, the spring, the sensor carrier, the magnetic component, and the sensor control device are in the proximal position.

10. The assembly of claim 9, wherein the adhesive patch comprises a third aperture, and wherein the sharp is configured to extend through the third aperture when the sharp carrier assembly, the sharp, the spring, the sensor carrier, the magnetic component, and the sensor control device are in the proximal position.

11. The assembly of claim 5, wherein the sensor carrier does not have one or more spring arms configured to retain the sensor control device.

12. The assembly of claim 5, wherein the one or more components of the sensor control device comprises one or more ferromagnetic components.

13. The assembly of claim 12, wherein the sensor control device further comprises a housing of the sensor control device defining an interior of the sensor control device, and wherein the one or more ferromagnetic components are disposed in the interior of the sensor control device.

14. The assembly of claim 13, wherein an adhesive force created by an adhesive property of the adhesive patch is greater than the magnetic force exerted by the magnetic component upon the one or more ferromagnetic components.

15. An assembly for insertion of a glucose sensor in a subject's body, the assembly comprising:
(1) an applicator, comprising:
an applicator housing defining an interior;
a sheath slidably coupled with the applicator housing and comprising a distal end configured for placement on a subject's skin;
a sharp carrier assembly coupled with a sharp;
a sensor carrier comprising a cavity formed by a plurality of walls, the plurality of walls including a proximal wall, a side wall, and a magnetic component, and wherein the cavity is configured to retain a sensor control device;
a spring comprising a distal end in contact with the sensor carrier; and
a cap configured to couple with a distal part of the applicator housing;
(2) the sensor control device configured to be worn on the subject's body, the sensor control device comprising:
an adhesive patch disposed on a bottom surface of the sensor control device and configured to adhere the sensor control device to the subject's skin;
the glucose sensor comprising:
a proximal portion configured to electrically couple with electronics;
a distal portion having a glucose-responsive enzyme, wherein the distal portion is configured to be positioned in a bodily fluid of the subject's body and to sense glucose in the subject's bodily fluid in a subcutaneous layer; and
the electronics comprising one or more processors, a memory, and communication circuitry configured to wirelessly transmit data according to a Bluetooth or a Bluetooth Low Energy protocol,
wherein the sharp carrier assembly, the sharp, the spring, the sensor carrier, the magnetic component, and the sensor control device are configured to advance a distance within the interior of the applicator housing, relative to the sheath, and in a linear direction from a proximal position to a distal position,
wherein, in the proximal position, a distal end of the glucose sensor is received in a portion of the sharp,
wherein the magnetic component is configured to exert a magnetic force upon one or more ferromagnetic components of the sensor control device when the sensor carrier and the sensor control device are in the proximal position,
wherein, in the distal position, the sensor control device is configured to adhere to the subject's skin via the adhesive patch, and disengage from the sensor carrier when the user pulls the applicator away from the skin, and
wherein the sensor control device further comprises a first aperture on a top surface of the housing of the sensor control device,
wherein the sensor control device further comprises a second aperture on the bottom surface of the sensor control device, and
wherein the sharp is configured to extend through the first aperture and the second aperture when the sharp carrier assembly, the sharp, the spring, the sensor carrier, the magnetic component, and the sensor control device are in the proximal position.

16. The assembly of claim 15, wherein the spring is configured to retract the sharp carrier assembly and the sharp in a proximal direction into the applicator after the sharp carrier assembly, the sharp, the spring, the sensor carrier, the magnetic component, and the sensor control device reach the distal position.

17. The assembly of claim 16, wherein the proximal portion of the glucose sensor comprises one or more electrical contacts.

18. The assembly of claim 17, wherein the glucose sensor further comprises a bent portion between the proximal portion of the glucose sensor and the distal portion of the glucose sensor.

19. The assembly of claim 18, wherein the proximal portion of the glucose sensor is perpendicular to the distal portion of the glucose sensor.

20. The assembly of claim 18, wherein the distal end of the glucose sensor and a distal portion of the sharp extend in a distal direction from the bottom surface of the sensor control device when the sharp carrier assembly, the sharp, the spring, the sensor carrier, the magnetic component, and the sensor control device are in the proximal position.
